(19) United States
(12) Patent Application Publication
(10) Pub. No.: US 2026/0325717 A1
(43) Pub. Date: Oct. 22, 2026

(54) COMPOSITIONS AND METHODS RELATED TO MICROGELS

(71) Applicant: The Regents of the University of California, Oakland, CA (US)

(72) Inventors: J. Kent LEACH, Davis, CA (US); Jeremy M. LOWEN, Davis, CA (US)

(21) Appl. No.: 19/100,490

(22) PCT Filed: Jun. 2, 2023
(86) PCT No.: PCT/US2023/024353

§ 371 Date: Jan. 31, 2025

Related U.S. Application Data
(60) Provisional application No. 63/394,691, filed on Aug. 3, 2022.

(51) Int. Cl. A61L 27/52; A61L 27/18; A61L 27/38; A61L 27/54; A61L 27/56

(52) U.S. Cl. CPC A61L 27/52; A61L 27/18; A61L 27/3834; A61L 27/54; A61L 27/56; A61L 2300/25

(57) ABSTRACT

The present disclosure provides compositions comprising a microgel scaffold formed by polyethylene glycol-vinyl sulfone (PEG-VS) and polyethylene gly-col-dithiol (PEG-DT), as well as methods of making thereof.

### CROSS-REFERENCE TO RELATED APPLICATION

This application is a U.S. National Phase Application Under 371 of International Application PCT/US2023/024353 filed Jun. 2, 2023, which claims priority to U.S. Provisional Application No. 63/394,691, filed Aug. 3, 2022, the disclosures of each of which are hereby incorporated by reference in their entireties for all purposes.

### STATEMENT AS TO RIGHTS TO INVENTIONS MADE UNDER FEDERALLY SPONSORED RESEARCH AND DEVELOPMENT

This invention was made with government support under Grant No. R01 DE025899, awarded by the National Institutes of Health (NIH). The government has certain rights in the invention.

### BACKGROUND

Polymeric scaffolds that are made of bulk hydrogels with a nanoporous mesh size are difficult for cells to infiltrate and also hinder biological activity. Microgels are appealing biomaterials for applications in regenerative medicine and tissue engineering due to their tunability, injectability, and ease in fabrication.

### BRIEF SUMMARY

In one aspect, the present disclosure provides a microgel scaffold comprising: a plurality of polyethylene glycol-vinyl sulfone (PEG-VS); and a plurality of polyethylene glycol-dithiol (PEG-DT) covalently linked to the plurality of PEG-VS, wherein the microgel scaffold has a compressive modulus of between 1 kPa and 30 kPa. In some embodiments, the microgel scaffold has a porosity of between 20 μm² and 100000 μm².

In another aspect, the disclosure provides a microgel scaffold comprising: a plurality of polyethylene glycol-vinyl sulfone (PEG-VS); and a plurality of polyethylene glycol-dithiol (PEG-DT) covalently linked to the plurality of PEG-VS, wherein the microgel scaffold has a porosity of between 20 μm² and 100000 μm². In some embodiments, the microgel scaffold has a compressive modulus of between 1 kPa and 30 kPa.

In some embodiments, the PEG-VS is selected from the group consisting of 3-arm PEG-VS, 4-arm PEG-VS, 6-arm PEG-VS, and 8-arm PEG-VS. In particular embodiments, the PEG-VS is an 8-arm PEG-VS.

In some embodiments, the PEG-VS has a molecular weight ranging from between 2 kDa and 40 kDa.

In some embodiments, the PEG-DT has a molecular weight ranging from between 0.6 kDa and 35 kDa.

In some embodiments, the between 40% and 80% (e.g., about 40%, 42%, 44%, 46%, 48%, 50%, 52%, 54%, 56%, 58%, 60%, 62%, 64%, 66%, 68%, 70%, 72%, 74%, 76%, 78%, or 80%) of the PEG-VS are linked to PEG-DT.

In some embodiments, the microgel scaffold further comprises a plurality of functional moieties conjugated to the PEG-VS. In certain embodiments, the functional moiety is a peptide (e.g., a cell adhesion peptide).

In some embodiments of the microgel scaffolds described herein, the microgel scaffolds further comprise live cells seeded within the microgel scaffold. In certain embodiments, the cells comprise human mesenchymal stem cells, induced pluripotent stem cells, embryonic stem cells, osteoblasts, immune cells, blood cells, adipocytes, or combinations thereof.

In another aspect, the disclosure provides a method of generating a microgel scaffold, the method comprising: forming a reaction mixture comprising a polyethylene glycol-vinyl sulfone (PEG-VS) and a polyethylene glycol-dithiol (PEG-DT); exposing the reaction mixture to ultraviolet (UV) irradiation for less than 2 minutes (e.g., 1 minute, 1.5 minutes, or 2 minutes) to anneal the PEG-VS and the PEG-DT, thereby forming the microgel scaffold. In some embodiments, the PEG-VS is selected from the group consisting of 3-arm PEG-VS, 4-arm PEG-VS, 6-arm PEG-VS, and 8-arm PEG-VS (e.g., 8-arm PEG-VS).

In some embodiments, the PEG-VS has a molecular weight ranging from between 2 kDa and 40 kDa. In some embodiments, the concentration of the PEG-VS in the reaction mixture is between 1 mM and 80 mM.

In some embodiments, the PEG-DT has a molecular weight ranging from between 0.6 kDa and 35 kDa. In certain embodiments, the concentration of the PEG-DT in the reaction mixture is between 1 mM and 300 mM.

In some embodiments of the methods described herein, between 40% and 80% (e.g., about 40%, 42%, 44%, 46%, 48%, 50%, 52%, 54%, 56%, 58%, 60%, 62%, 64%, 66%, 68%, 70%, 72%, 74%, 76%, 78%, or 80%) of the PEG-VS are linked to PEG-DT.

In some embodiments of the methods described herein, prior to the exposing step, a photoinitiator is added to the reaction mixture. In certain embodiments, the concentration of the photoinitiator in the reaction mixture is between 0.05% w/v and 0.8% w/v. In certain embodiments, the UV irradiation has a power within the range between 2 mW/cm² and 20 mW/cm².

In further embodiments of the methods described herein, the method further comprises, after the exposing step, attaching a functional moiety to the microgel scaffold. In some embodiments, the functional moiety is a peptide, e.g., a cell adhesion peptide.

In some embodiments of the methods described herein, the method further comprises, after the exposing step, centrifuging the microgel scaffold at different speeds to achieve a desired porosity of the microgel scaffold.

In some embodiments, the method further comprises, after the exposing step, seeding the microgel scaffold with live cells. Examples of cells include, but are not limited to, human mesenchymal stem cells, induced pluripotent stem cells, embryonic stem cells, osteoblasts, immune cells, blood cells, adipocytes, or combinations thereof.

### Definitions

The term “microgel” refers to a three-dimensional hydrogel particle that generally has a diameter between 10 μm-500 μm.

The term “microgel scaffold” refers to a network or scaffolding of microgels in which the microgels are crosslinked to each other by way of, for example, covalent bonds.

The term “photoinitiator” refers to a compound that initiates a polymerization process after irradiation. In some embodiments, the photoinitiator can generate acid (a photo-acid generator or PAG) or a radical, among other initiating species. The acid, radical, or other species, then initiates a polymerization.

The term “biocompatible” refers to a material, composition, device, or method that does not have toxic or injurious effects on biological systems. In some medical applications, a biocompatible material, composition, device, or method does not have toxic or injurious effects on a treated subject.

### BRIEF DESCRIPTION OF THE DRAWINGS

FIGS. 1A-1E: Microgel annealing process. A) The ratio of PEG-VS to PEG-DT can be stoichiometrically controlled so that arms are left free for annealing. B) Microgels are mixed with additional PEG-DT before annealing, spun down, and exposed to UV light to anneal. C) Different solutions were tested for the strongest annealing between microgels. D) A comparison of different PEG-DT concentrations in the annealing solution. A 5 mM solution was sufficient for maximum annealing. Scale bar represents 200 μm. E) Constructs over 1 cm in thickness were annealed using UV light. Statistics: 2-way ANOVA, Tukey's multiple comparisons test. N≥4. *p<0.05, **p<0.01, and ***p<0.001.

FIGS. 2A-2G: Mechanical properties of microgels and cryopreservation. A) MicroTesting of large microgel. B) Compressive modulus of bulk gels versus microgels. C) Mesh size difference in 10 kDa versus 20 kDa PEG-VS. D) Compressive modulus of microgels after being frozen. E) Addition of fluorescent peptide to verify successful peptide addition. Scale bar represents 100 μm. F) Images of cell spreading on frozen and fresh gels compared to non-spread cells on negative control. Scale bar represents 200 μm. G) Alamar blue showing positive metabolic activity for all conditions. Statistics: (B) 2-way ANOVA, Tukey's multiple comparisons test. (D,G) Ordinary one-way ANOVA, Tukey's multiple comparisons test. N≥3. *p<0.05, **p<0.01, and ***p<0.001.

FIGS. 3A-3E: Modeling void space in between microgels. A) Custom MATLAB code detects and measures microgel diameters. Scale bar represents 500 μm. B) Histograms of small and large microgel diameters. C) Modeling microgel annealing in MATLAB. D) Measuring void space area in FIJI. E) Predicted void space area vs experimentally measured void space area. Statistics: Ordinary one-way ANOVA, Tukey's multiple comparisons test. N≥100. *p<0.05, **p<0.01, and ***p<0.001.

FIGS. 4A-4D: Microgels as an aggregate-forming platform. A) MSC aggregate formation in large and small microgels over 48 hours. More monodisperse cells are seen in small microgels. MSCs circled in white. B) Quantification of aggregate size demonstrates how larger aggregates form in large microgel scaffolds. C) DAPI/Phalloidin stain reveals the formation of aggregates throughout the large microgel scaffold after 48 hours. D) Live/dead stain of aggregates after retrieval from large microgel scaffolds. Scale bars represent 100 μm. Statistics: two-way ANOVA, Sidak's multiple comparisons test. n≥46. p<0.05 was considered significant. Data points with different letters are significantly different from one another.

FIGS. 5A-5D: Spheroids spread rapidly in large and small microgel scaffolds. A) DAPI/Phalloidin stains illustrate that void space inherent in microgel scaffolds facilitates rapid spreading in large and small microgel scaffolds. Inset scale bars represent 200 μm. Primary picture scale bars represent 1 mm. B) Migration distance did not differ between microgel scaffolds. C) Cell spreading is denser in small microgel scaffolds versus large microgel scaffolds at Day 1 and Day 7. D) alamarBlue assay confirms that both scaffolds promote high cell metabolic activity. Statistics: (B,D) Two-way ANOVA, Tukey's multiple comparisons test. (C) Multiple unpaired Student's t tests. n≥3. *p<0.05, **p<0.01, and ***p<0.001.

FIGS. 6A-6G: Microgel size influences macrophage polarization. A) Macrophages (red) in small microgel scaffolds are trapped between them and exhibit an elongated cell morphology (white arrows). Macrophages in large microgel scaffolds are more rounded and can be found in clusters. B,E) Cell viability is similar in both small and large microgel scaffolds. C,F) M1 polarization is trending toward being higher in the smaller microgel scaffolds. D,G) M2 polarization is greater in large microgel scaffolds. Scale bars represent 100 μm. Statistics: Unpaired Student's t-tests. n≥4. *p<0.05, **p<0.01.

FIGS. 7A-7C: Subfascial implantation of microgel scaffolds. A) Microgel scaffolds were implanted in four locations per mouse. B) PDMS mold that was loaded with microgels and implanted subfascially. C) H&E and Masson's trichrome staining illustrate greater cell infiltration into large microgel scaffolds. Fibrous tissue is present on the outside of some of the PDMS molds (white arrows). Primary scale bars represent 100 μm. Inset scale bars represent 20 μm.

FIG. 8: Storage modulus of microgel scaffolds increases over time when exposed to UV light.

### DETAILED DESCRIPTION

The extracellular matrix of cells plays a vital role in processes such as differentiation, angiogenesis, proliferation, invasion, and wound repair. To successfully undergo these processes cells must be able to remodel their surrounding environment to create space for migration and protein deposition. Described herein are highly tunable microgels with size, stiffness, and biochemical presentation modified to influence cell fate. Ultraviolet light is utilized to anneal the microgels together, creating a scaffold with microporous void space throughout. By altering microgel diameter, it is possible to control void space size and therefore the amount of cell spreading and aggregation throughout the annealed microgel scaffold. This void space allows for rapid infiltration without cells having to remodel the surrounding environment or wait for a porogen to dissolve. The use of ultraviolet light allows for microgels to be noninvasively injected into a desired mold or wound defect before annealing, and microgels of different properties can combined to create a heterogenous scaffold. This approach is clinically relevant given its tunability to many tissue types and fast annealing time, e.g., under 1 minute.

### I. INTRODUCTION

Tunable and noninvasive scaffolds to direct cell fate are desired in many tissue engineering applications such as wound healing, organoid systems, and drug delivery. As the understanding of tissue complexity increases so does the demand for heterogeneous biomaterials. Microgels are an emerging tool which fulfill this roll given their modularity, injectability, and range of fabrication techniques. A plethora of studies have harnessed microgels to study cell behavior in response to stiffness, degradability, and biochemical cues. A range of materials are commonly used to synthesize microparticles including alginate, poly(ethylene) glycol (PEG), and hyaluronic acid. Given the vast amount of materials and tunability it is possible to synthesize microgels to meet a variety of scaffold specifications.

Historically, polymeric scaffolds are composed of bulk hydrogels that exhibit a nanoporous mesh size. This pore size prevents infiltration by cells and hinders biological activity. As a result, the inclusion of a matrix metalloproteinase (MMP)-sensitive crosslinker is often used to enable cells to degrade and remodel their surrounding environment. Cells will secrete MMPs in response to environmental stimuli, such as macrophages secreting MMP-9 in response to infection. An advantage to microparticle-based scaffold is the void space which inherently exists between the particles. This permits cells to immediately migrate without having to remodel their surrounding environment. Previous work has illustrated that human dermal fibroblasts proliferated over twice as much in a microgel scaffold compared to a bulk hydrogel scaffold.

Recent work has demonstrated the modularity of microgels by combining microgels of different properties to create gradients of stiffness and degradability. While creating spatially modified hydrogels is often difficult due to the use of homogenously mixed precursor solutions, microgels overcome this challenge by decoupling scaffold formation from hydrogel crosslinking. It is also necessary to create an incision to insert bulk hydrogels a defect site subcutaneously. Microgel scaffolds overcome this limitation with injectable microporous scaffolds out-performing their non-porous controls in wound healing. Microgels are also able to maintain their spatial orientation when loaded and extruded from a syringe, and have the advantage of naturally filling up the dimensions of the defect site.

While many microgel platforms rely on chemical assembly methods such as enzymatic catalysis or click chemistry, demonstrated herein is an ultraviolet (UV) method of annealing microgels. This method is effective for microgels of various sizes, e.g., ranging from about 45 μm to about 140 μm in diameter and requires as little as one minute for annealing. Microgels spanning from about 10 kPa to 80 kPa were generated. Their compressive modulus was compared to to bulk gels using a MicroTester. Further, the compressive modulus and bioactivity of the microgels remained constant after being cryopreserved at −20° C. A custom MATLAB code was also developed to predict void space diameter based on theoretical and observed microgel diameters. The ability to alter porosity was utilized to influence macrophage polarization. This platform's ability to predictability alter cell spreading and aggregation was also demonstrated. The ease of recovering cells from this system was also utilized to run flow cytometry to examine polarization state of the macrophages. Furthermore, the conditioned media from the macrophage seeded scaffolds was then collected to influence mesenchymal stem cell (MSC) differentiation. The ease of loading cells, cryopreservability, and short annealing time make this a promising therapeutic for the clinic.

The present disclosure provides a microgel scaffold that is made from a plurality of polyethylene glycol-vinyl sulfone (PEG-VS) and a plurality of polyethylene glycol-dithiol (PEG-DT) covalently linked to the plurality of PEG-VS. The microgel scaffold described herein has a compressive modulus of between 1 kPa and 30 kPa and/or a porosity of between 20 μm² and 100000 μm². The disclosure also provides methods of making the microgel scaffold that utilizes ultraviolet (UV) irradiation.

### II. MICROGEL SCAFFOLD

The microgel scaffold of the present disclosure comprises a plurality of polyethylene glycol-vinyl sulfone (PEG-VS); and a plurality of polyethylene glycol-dithiol (PEG-DT) covalently linked to the plurality of PEG-VS. In some embodiments, the PEG-VS used can contain different numbers of arms depending on the desired degree of crosslinking between PEG-VS and PEG-DT. In some embodiments, the PEG-VS is selected from the group consisting of 3-arm PEG-VS, 4-arm PEG-VS, 6-arm PEG-VS, and 8-arm PEG-VS. In certain embodiments, the PEG-VS is an 8-arm PEG-VS. In certain embodiments, the PEG-VS has a molecular weight ranging from between 2 kDa and 40 kDa (e.g., between 2 kDa and 35 kDa, between 2 kDa and 30 kDa, between 2 kDa and 25 kDa, between 2 kDa and 20 kDa, between 2 kDa and 15 kDa, between 2 kDa and 10 kDa, between 2 kDa and 5 kDa, between 5 kDa and 40 kDa, between 10 kDa and 40 kDa, between 15 kDa and 40 kDa, between 20 kDa and 40 kDa, between 25 kDa and 40 kDa, between 30 kDa and 40 kDa, or between 35 kDa and 40 kDa; 2 kDa, 4 kDa, 6 kDa, 8 kDa, 10 kDa, 12 kDa, 14 kDa, 16 kDa, 18 kDa, 20 kDa, 22 kDa, 24 kDa, 26 kDa, 28 kDa, 30 kDa, 32 kDa, 34 kDa, 36 kDa, 38 kDa, or 40 kDa).

In some embodiments, the PEG-DT has a molecular weight ranging from between 0.6 kDa and 35 kDa (e.g., between 0.6 kDa and 30 kDa, between 0.6 kDa and 25 kDa, between 0.6 kDa and 20 kDa, between 0.6 kDa and 15 kDa, between 0.6 kDa and 10 kDa, between 0.6 kDa and 5 kDa, between 0.6 kDa and 3 kDa, between 0.6 kDa and 1 kDa, between 1 kDa and 35 kDa, between 5 kDa and 35 kDa, between 10 kDa and 35 kDa, between 15 kDa and 35 kDa, between 20 kDa and 35 kDa, between 25 kDa and 35 kDa, or between 30 kDa and 35 kDa; 0.6 kDa, 1 kDa, 5 kDa, 10 kDa, 15 kDa, 20 kDa, 25 kDa, 30 kDa, or 35 kDa).

As described in detail further herein, the microgel scaffolds described herein are highly tunable and can be constructed with different sizes of porosity within the microgel scaffolds. The porosity of the microgel scaffolds can be tuned depending on the use purpose and allows rapid cell infiltration into the microgel scaffolds without the cells needing to remodel their surrounding environment. In some embodiments, the microgel scaffold comprises a porosity of between 20 μm² and 100000 μm² (e.g., between 20 μm² and 90000 μm², between 20 μm² and 80000 μm², between 20 μm² and 70000 μm², between 20 μm² and 60000 μm², between 20 μm² and 50000 μm², between 20 μm² and 40000 μm², between 20 μm² and 30000 μm², between 20 μm² and 20000 μm², between 20 μm² and 10000 μm², between 20 μm² and 9000 μm², between 20 μm² and 8000 μm², between 20 μm² and 7000 μm², between 20 μm² and 6000 μm², between 20 μm² and 5000 μm², between 20 μm² and 4000 μm², between 20 μm² and 3000 μm², between 20 μm² and 2000 μm², between 20 μm² and 1000 μm², between 20 μm² and 900 μm², between 20 μm² and 800 μm², between 20 μm² and 700 μm², between 20 μm² and 600 μm², between 20 μm² and 500 μm², between 20 μm² and 400 μm², between 20 μm² and 300 μm², between 20 μm² and 200 μm², between 20 μm² and 100 μm², between 20 μm² and 90 μm², between 20 μm² and 80 μm², between 20 μm² and 70 μm², between 20 μm² and 60 μm², between 20 μm² and 50 μm², between 20 μm² and 40 μm², or between 20 μm² and 30 μm²).

In some embodiments, the microgel scaffold comprises a porosity of between 30 μm² and 100000 μm² (e.g., between 40 μm² and 100000 μm², between 50 μm² and 100000 μm², between 60 μm² and 100000 μm², between 70 μm² and 100000 μm², between 80 μm² and 100000 μm², between 90 μm² and 100000 μm², between 100 μm² and 100000 μm², between 200 μm² and 100000 μm², between 300 μm² and 100000 μm², between 400 μm² and 100000 μm², between 500 μm² and 100000 μm², between 600 μm² and 100000 μm², between 700 μm² and 100000 μm², between 800 μm² and 100000 μm², between 900 μm² and 100000 μm², between 1000 μm² and 100000 μm², between 2000 μm² and 100000 μm², between 3000 μm² and 100000 μm², between 4000 μm² and 100000 μm², between 5000 μm² and 100000 μm², between 6000 μm² and 100000 μm², between 7000 μm² and 100000 μm², between 8000 μm² and 100000 μm², between 9000 μm² and 100000 μm², between 10000 μm² and 100000 μm², between 20000 μm² and 100000 μm², between 30000 μm² and 100000 μm², between 40000 μm² and 100000 μm², between 50000 μm² and 100000 μm², between 60000 μm² and 100000 μm², between 70000 μm² and 100000 μm², between 80000 μm² and 100000 μm², or between 90000 μm² and 100000 μm²).

Moreover, in some instances, the microgel scaffold described herein can have a compressive modulus of between 1 kPa and 30 kPa (e.g., between 1 kPa and 28 kPa, between 1 kPa and 26 kPa, between 1 kPa and 24 kPa, between 1 kPa and 22 kPa, between 1 kPa and 20 kPa, between 1 kPa and 18 kPa, between 1 kPa and 16 kPa, between 1 kPa and 14 kPa, between 1 kPa and 12 kPa, between 1 kPa and 10 kPa, between 1 kPa and 8 kPa, between 1 kPa and 6 kPa, between 1 kPa and 4 kPa, between 1 kPa and 2 kPa, between 2 kPa and 30 kPa, between 4 kPa and 30 kPa, between 6 kPa and 30 kPa, between 8 kPa and 30 kPa, between 10 kPa and 30 kPa, between 12 kPa and 30 kPa, between 14 kPa and 30 kPa, between 16 kPa and 30 kPa, between 18 kPa and 30 kPa, between 20 kPa and 30 kPa, between 22 kPa and 30 kPa, between 24 kPa and 30 kPa, between 26 kPa and 30 kPa, or between 28 kPa and 30 kPa; 1 kPa, 2 kPa, 4 kPa, 6 kPa, 8 kPa, 10 kPa, 12 kPa, 14 kPa, 16 kPa, 18 kPa, 20 kPa, 22 kPa, 24 kPa, 26 kPa, 28 kPa, or 30 kPa).

Further, the PEG-VS in the microgel scaffold described herein can be modified with different functional moieties. In some embodiments, the functional moieties are used to direct and influence cell seeding, attachment, spreading, and/or growth as the cells are injected into the microgel scaffold. In other embodiments, the functional moieties can also direct and influence cell fate and differentiation. Examples of functional moieties that can be attached to the PEG-VS include, but are not limited to, a peptide, an oligonucleotide, a fluorescent label, and/or a carbohydrate. In certain embodiments, the PEG-VS in the microgel scaffold can be modified with a peptide. In particularly, the peptide can be a cell adhesion peptide, which promotes cell adhesion and is often derived from extracellular matrix glycoproteins such as laminin, fibronectin, and collagen. Examples of cell adhesion peptides can be attached to PEG-VS include, but are not limited to, RGDSPGERCG (SEQ ID NO:1), HAVDIGGGC (SEQ ID NO:2), LNIVSVNGRH (SEQ ID NO:3), DNRIRLQAK (SEQ ID NO:4), KATPMLKMRTSFHGCIK (SEQ ID NO:5), KEGYKVRLDLNITLEFRTTSK (SEQ ID NO:6), KNLEISRSTFDLLRNSYGVRK (SEQ ID NO:7), KQNCLSSRASFRGCVRNLRLSR (SEQ ID NO:8), KQKCLRSQTSFRGCLRKLALIK (SEQ ID NO:9), CRNRGRCNSSLFQVRSRKLLSA (SEQ ID NO:10), KQCLKSQRSFTRGLCRLKAKIL (SEQ ID NO:11), KLLISRARKQAASIK (SEQ ID NO:12), LERKYENDQKYLEDKA (SEQ ID NO:13), and VEKRGDREEA (SEQ ID NO: 14). In particular embodiments, the cell adhesion peptide has the sequence of RGDSPGERCG (SEQ ID NO:1). In particular embodiments, the cell adhesion peptide has the sequence of HAVDIGGGC (SEQ ID NO:2). Additional cell adhesion peptides are described in, e.g., Huettner et al., Trends Biotechnol 2018 Apr;36(4):372-383; Ruoslahti Annu Rev Cell Dev Biol 1996; 12:697-715; and LeBaron and Athanasiou Tissue Eng 2000 Apr;6(2):85-103.

The microgel scaffold of the present disclosure can be seeded with different types of cells. The cells can be, for example and without limitation, human mesenchymal stem cells (hMSCs), induced pluripotent stem cells (iPSCs), embryonic stem cells (ESCs), osteoblasts and osteoblast precursors (e.g, MC3T3 cells), immune cells (e.g., macrophages), blood cells, adipocytes, and others. In particular embodiments, the cells seeded in the microgel scaffold are live cells. In some embodiments, the seeding of the microgel scaffold with cells can occur prior to the injecting of the microgel scaffold into its destination, e.g., tissue and/or bone defects. In other embodiments, the seeding of the microgel scaffold with cells can occur subsequent to the injecting of the microgel scaffold into its destination, e.g., tissue and/or bone defects.

### III. METHODS OF GENERATION

Provided herein are also methods of generating the microgel scaffolds described herein using a rapid ultraviolet (UV) irradiation process. First, a reaction mixture comprising a plurality of polyethylene glycol-vinyl sulfone (PEG-VS) and a plurality of polyethylene glycol-dithiol (PEG-DT) is formed. The reaction mixture is then exposed to UV irradiation for less than 2 minutes to anneal the PEG-VS and the PEG-DT, thereby forming the microgel scaffold.

In some embodiments, the UV irradiation of the reaction mixture to form the microgel scaffold is about 30 seconds, 40 seconds, or 50 seconds. In some embodiments, the UV irradiation of the reaction mixture to form the microgel scaffold is about 60 seconds (1 minute), 70 seconds, 80 seconds, 90 seconds, 100 seconds, 110 seconds, or 120 seconds (2 minutes). In certain embodiments, the power of the UV irradiation is between 2 and 20 mW/cm², e.g., 2 mW/cm², 4 mW/cm², 6 mW/cm², 8 mW/cm², 10 mW/cm², 12 mW/cm², 14 mW/cm², 16 mW/cm², 18 mW/cm², or 20 mW/cm². In particular embodiments, the methods of generating the microgel scaffolds described herein use 1 minute UV irradiation at 20 mW/cm². In particular embodiments, the methods of generating the microgel scaffolds described herein use 1.5 minute UV irradiation at 20 mW/cm². In particular embodiments, the methods of generating the microgel scaffolds described herein use 2 minutes UV irradiation at 20 mW/cm².

In certain embodiments, the concentration of the PEG-VS in the reaction mixture is between 1 mM and 80 mM (e.g., between 1 mM and 70 mM, between 1 mM and 60 mM, between 1 mM and 50 mM, between 1 mM and 40 mM, between 1 mM and 30 mM, between 1 mM and 20 mM, between 1 mM and 10 mM, between 1 mM and 5 mM, between 5 mM and 80 mM, between 10 mM and 80 mM, between 20 mM and 80 mM, between 30 mM and 80 mM, between 40 mM and 80 mM, between 50 mM and 80 mM, between 60 mM and 80 mM, or between 70 mM and 80 mM; 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, or 80 mM).

In certain embodiments, the concentration of the PEG-DT in the reaction mixture is between 1 mM and 300 mM (e.g., between 1 mM and 280 mM, between 1 mM and 260 mM, between 1 mM and 240 mM, between 1 mM and 220 mM, between 1 mM and 200 mM, between 1 mM and 180 mM, between 1 mM and 160 mM, between 1 mM and 140 mM, between 1 mM and 120 mM, between 1 mM and 100 mM, between 1 mM and 80 mM, between 1 mM and 60 mM, between 1 mM and 40 mM, between 1 mM and 20 mM, between 1 mM and 10 mM, between 10 mM and 300 mM, between 20 mM and 300 mM, between 40 mM and 300 mM, between 60 mM and 300 mM, between 80 mM and 300 mM, between 100 mM and 300 mM, between 120 mM and 300 mM, between 140 mM and 300 mM, between 160 mM and 300 mM, between 180 mM and 300 mM, between 200 mM and 300 mM, between 220 mM and 300 mM, between 240 mM and 300 mM, between 260 mM and 300 mM, or between 280 mM and 300 mM). In some embodiments, the concentration of the PEG-DT in the reaction mixture is about 1 mM, 10 mM, 20 mM, 40 mM, 60 mM, 80 mM, 100 mM, 120 mM, 140 mM, 160 mM, 180 mM, 200 mM, 220 mM, 240 mM, 260 mM, 280 mM, or 300 mM).

The size of the porosity, or void space, within the microgel scaffolds can be changed easily by incorporating PEG-VS of different arms, as well as altering the amount of PEG-DT and the ratio of PEG-VS to PEG-DT in the reaction mixture to tune the degree of crosslinking of PEG-VS and PEG-DT. In further embodiments, after the exposing the reaction mixture containing PEG-VS and PEG-DT to UV irradiation, the microgel scaffold can be centrifuged at different speeds to achieve a desired porosity of the microgel scaffold. In some embodiments, for a microgel scaffold containing large individual microgels (e.g., microgels that are at least 100 μm (e.g., at least 120, 140, 160, 180, 200, 220, 240, 260, 280, or 300 μm)), a higher centripetal force (e.g., a centripetal force of at least 15,000 g) can be used to reduce the porosity of the microgel scaffold.

In some embodiments of the methods of generating the microgel scaffolds described herein, sacrificial materials can be added to increase the porosity of the microgel scaffold. Examples of sacrificial materials include, but are not limited to, salts, sugars, paraffin, and gelatin. In some embodiments, sacrificial materials can be removed by immersing the scaffold in a solvent which dissolves the porogen, or increasing the temperature to dissolve the porogen.

In some embodiments, a photoinitiator is added to the reaction mixture prior to exposing the reaction mixture to UV irradiation. The concentration of photoinitiator in the reaction mixture to generate the microgel scaffolds can be within the range between 0.05% w/v and 0.8% w/v. The concentration of photoinitiator can be within the range between 0.05% w/v and 0.6% w/v, between 0.05% w/v and 0.5% w/v, between 0.05% w/v and 0.4% w/v, between 0.05% w/v and 0.3% w/v, between 0.05% w/v and 0.2% w/v, or between 0.05% w/v and 0.1% w/v. The concentration of photoinitiator can be within the range between 0.1% w/v and 0.8% w/v, between 0.2% w/v and 0.8% w/v, between 0.3% w/v and 0.8% w/v, between 0.4% w/v and 0.8% w/v, between 0.5% w/v and 0.8% w/v, between 0.6% w/v and 0.8% w/v, or between 0.7% w/v and 0.8% w/v.

Different photoinitiators are sensitive to different wavelenghts, such as wavelengths in the UV spectrum (e.g., from about 100 to about 400 nm) and wavelengths in the visible light spectrum (e.g., from about 400 to about 800 nm). Examples of photoinitiators include, but are not limited to, eosin Y, VA-086, riboflavin, carboxylated camphorquinone, rose bengal, erythrosine, WSPI, BDEA, 2PCK, G2CK, Irgacure 2959, Irgacure 184, Irgacure 651, Irgacure 369, Irgacure 907, PEGDA, MeHA, GelMA, MAPO (TPO), TPO-Na, LAP, BAPO, BAPO-Ona, and BAPO-OLi Photoinitiators are also described in, e.g., Tomal and Ortyl, Polymers (Basel). 2020 May; 12(5): 1073; Shukla et al., Polymers (Basel). 2021 Aug. 12; 13(16):2694; and Chuilan et al., Biomacromolecules. 2021 May 10;22(5):1795-1814.

In certain embodiments, the methods of generating microgel scaffolds described herein use a plurality of PEG-VS selected from the group consisting of 3-arm PEG-VS, 4-arm PEG-VS, 6-arm PEG-VS, and 8-arm PEG-VS. In particular embodiments, the PEG-VS is an 8-arm PEG-VS. The PEG-VS used in the methods can have a molecule weight ranging from between 2 kDa and 40 kDa (e.g., between 2 kDa and 35 kDa, between 2 kDa and 30 kDa, between 2 kDa and 25 kDa, between 2 kDa and 20 kDa, between 2 kDa and 15 kDa, between 2 kDa and 10 kDa, between 2 kDa and 5 kDa, between 5 kDa and 40 kDa, between 10 kDa and 40 kDa, between 15 kDa and 40 kDa, between 20 kDa and 40 kDa, between 25 kDa and 40 kDa, between 30 kDa and 40 kDa, or between 35 kDa and 40 kDa; 2 kDa, 4 kDa, 6 kDa, 8 kDa, 10 kDa, 12 kDa, 14 kDa, 16 kDa, 18 kDa, 20 kDa, 22 kDa, 24 kDa, 26 kDa, 28 kDa, 30 kDa, 32 kDa, 34 kDa, 36 kDa, 38 kDa, or 40 kDa).

In certain embodiments, the methods of generating microgel scaffolds described herein use a plurality of PEG-DT, in which the PEG-DT has a molecular weight ranging from between 0.6 kDa and 35 kDa (e.g., between 0.6 kDa and 30 kDa, between 0.6 kDa and 25 kDa, between 0.6 kDa and 20 kDa, between 0.6 kDa and 15 kDa, between 0.6 kDa and 10 kDa, between 0.6 kDa and 5 kDa, between 0.6 kDa and 3 kDa, between 0.6 kDa and 1 kDa, between 1 kDa and 35 kDa, between 5 kDa and 35 kDa, between 10 kDa and 35 kDa, between 15 kDa and 35 kDa, between 20 kDa and 35 kDa, between 25 kDa and 35 kDa, or between 30 kDa and 35 kDa; 0.6 kDa, 1 kDa, 5 kDa, 10 kDa, 15 kDa, 20 kDa, 25 kDa, 30 kDa, or 35 kDa).

Further, provided herein are methods of generating a microgel scaffold comprising: forming a reaction mixture comprising a plurality of an 8-arm PEG-VS and a plurality of a PEG-DT; exposing the reaction mixture to UV irradiation for less than 2 minutes (e.g., 1 minute, 1.5 minutes, or 2 minutes) to anneal the PEG-VS and the PEG-DT, thereby forming the microgel scaffold. In certain embodiments, the PEG-DT used in the methods is about 3.5 kDa. In certain embodiments, the PEG-DT used in the methods is about 4-5 mM (e.g., 5 mM). In some embodiments of the methods, an eosin Y photoinitiator is added to the reaction mixture prior to exposing the reaction mixture to UV irradiation. In other embodiments of the methods, a VA-086 photoinitiator is added to the reaction mixture prior to exposing the reaction mixture to UV irradiation.

### IV. METHODS OF USE

In some embodiments, the microgel scaffolds described herein are readily amenable to use with different of cell types and for a variety of clinical relevant applications. The high tunability of the microgel scaffolds described herein makes the microgel scaffolds easily tailored to a diverse range of cell types of different sizes and attachment, migration, and growth patterns. As described herein, the size of the porosity, or void space, within the microgel scaffolds can be changed easily by incorporating PEG-VS of different arms, as well as altering the amount of PEG-DT and the ratio of PEG-VS to PEG-DT in the reaction mixture to tune the degree of crosslinking of PEG-VS and PEG-DT.

Typically, cells can be seeded in microgel scaffolds described herein are mammalian cells, although the cells may be from different species (e.g., humans, mice, rats, primates, pigs, and the like). The cells can be primary cells, or they may be derived from an established cellline. Cells can be from multiple donor types, can be progenitor cells, tumor cells, and the like. In other embodiments, the cells are freshly isolated cells (for example, seeded within 24 hours of isolation), e.g., freshly isolated cells from donor organs. In certain embodiments, the cells are isolated from individual donor organs and an assembled tissue construct is specific for that donor.

Further, cell types which may be seeded in the microgel scaffolds described herein include pancreatic cells (alpha, beta, gamma, delta), enterocytes, renal epithelial cells, astrocytes, muscle cells, brain cells, neurons, glia cells, respiratory epithelial cells, lymphocytes, erythrocytes, blood-brain barrier cells, kidney cells, cancer cells, normal or transformed fibroblasts, liver progenitor cells, oval cells, adipocytes, osteoblasts, osteoclasts, myoblasts, beta-pancreatic islets cells, stem cells (e.g., embryonic stem cells, hematopoietic stem cells, mesenchymal stem cells, endothelial stem cells, etc.), myocytes, keratinocytes, and indeed any cell type that adheres to a substrate.

In some embodiments, the microgel scaffolds described herein can include peptides or proteins from the extracellular matrix (ECM), i.e., an ECM peptide or protein conjugated to the PEG-VS. ECM peptides and proteins found in the cell's native microenvironment are useful in maintaining attachment, growth, migration, and/or function of the cell. An ECM peptide or protein can be a cell adhesion peptide or protein that is specific to the cell type to be seeded in the microgel scaffolds. Examples of cell adhesion peptides can be attached to PEG-VS include, but are not limited to, RGDSPGERCG (SEQ ID NO:1), HAVDIGGGC (SEQ ID NO:2), LNIVSVNGRH (SEQ ID NO:3), DNRIRLQAK (SEQ ID NO:4), KATPMLKMRTSFHGCIK (SEQ ID NO:5), KEGYKVRLDLNITLEFRTTSK (SEQ ID NO:6), KNLEISRSTFDLLRNSYGVRK (SEQ ID NO:7), KQNCLSSRASFRGCVRNLRLSR (SEQ ID NO:8), KQKCLRSQTSFRGCLRKLALIK (SEQ ID NO:9), CRNRGRCNSSLFQVRSRKLLSA (SEQ ID NO:10), KQCLKSQRSFTRGLCRLKAKIL (SEQ ID NO:11), KLLISRARKQAASIK (SEQ ID NO:12), LERKYENDQKYLEDKA (SEQ ID NO:13), and VEKRGDREEA (SEQ ID NO: 14). In particular embodiments, the cell adhesion peptide has the sequence of RGDSPGERCG (SEQ ID NO:1). In particular embodiments, the cell adhesion peptide has the sequence of HAVDIGGGC (SEQ ID NO:2). Other ECM peptides and proteins include, but are not limited to, collagen I, collagen III, collagen IV, laminin, and fibronectin.

In certain embodiments, the cells that can be seeded into the microgel scaffolds described herein include, but are not limited to, human mesenchymal stem cells, induced pluripotent stem cells, embryonic stem cells, osteoblasts, immune cells (e.g., macrophages), blood cells (e.g., redblood cells), adipocytes, or combinations thereof.

In some embodiments, the microgel scaffolds described herein can be injected into the desired mold or wound defect before annealing using UV irradiation. In other embodiments, the microgel scaffolds described herein can be injected into the desired mold or wound defect after annealing using UV irradiation. Particularly, in some embodiments, the cells can be seeded into the microgel scaffolds after the microgel scaffolds are injected into the desired mold or would defect. In other embodiments, the cells can be seeded into the microgel scaffolds before the microgel scaffolds are injected into the desired mold or would defect.

Depending on the desired mold, wound defect, and/or cell types needed, microgel scaffolds of different porosity and/or with different cell types seeded can be combined to construct a heterogeneous microgel scaffolds.

### Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1—Synthesis and Annealing of Microgels and Viability Study

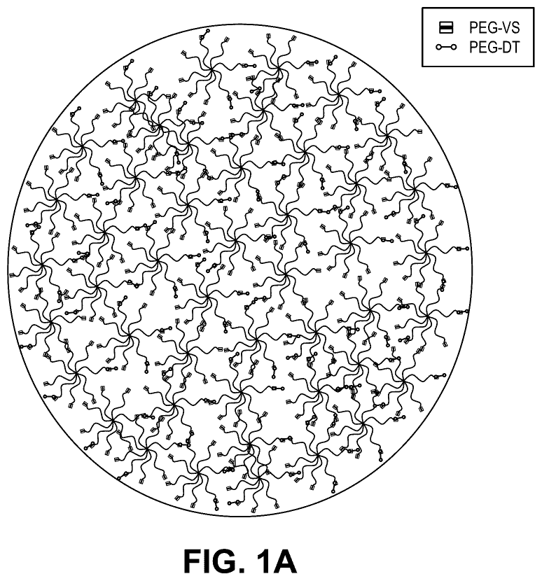

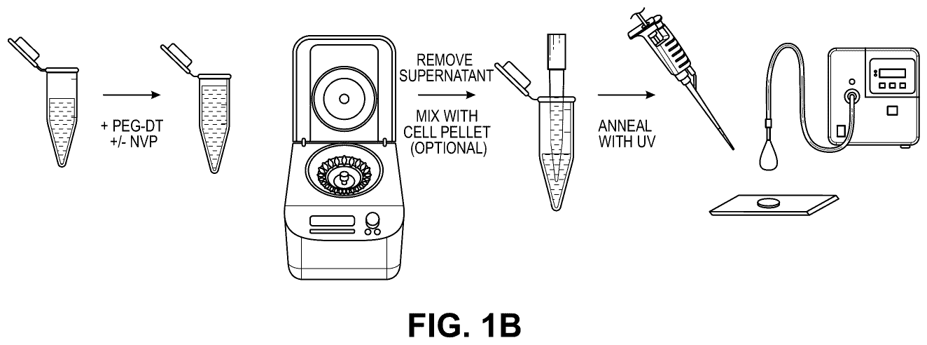

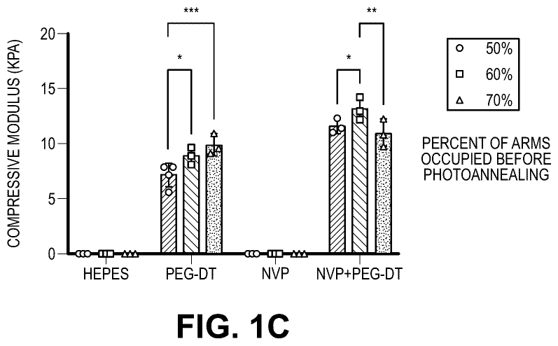

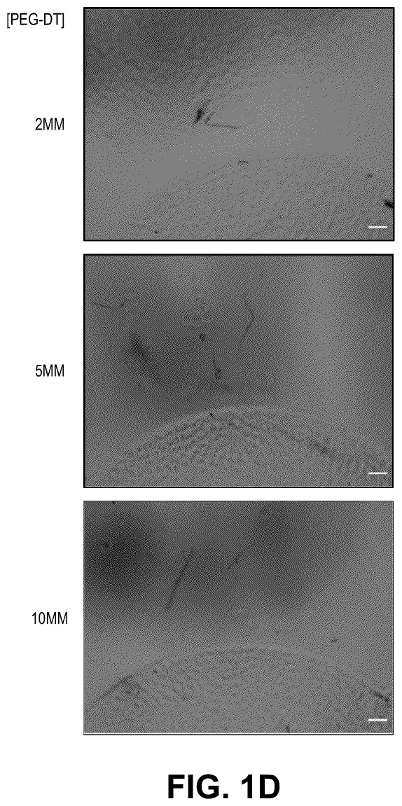

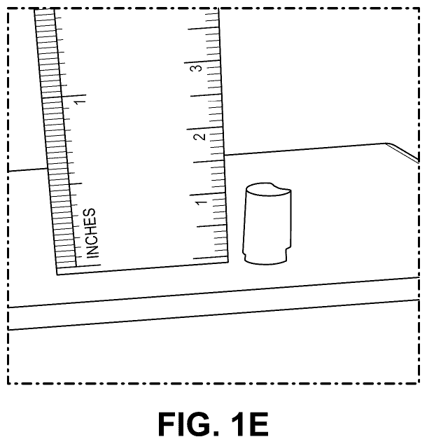

Microgels were synthesized using a previously established high-throughput microfluidic device. To facilitate microgel annealing we produced microgels with only a fraction of the arms crosslinked to enable the remaining free arms to be used for photoannealing (FIG. 1A). We stoichiometrically controlled the number of free arms by altering the ratio of 8-arm PEG-vinyl sulfone (PEG-VS) to the crosslinker PEG-dithiol (PEG-DT). By using the vinyl sulfone moiety for both microgel crosslinking and annealing we avoid the use of additional complex or expensive reagents used in methods such as enzymatic catalysis or host-guest interactions. The use of an 8-arm PEG allows us to fine tune the number of arms used for crosslinking while leaving arms available for biofunctionalization such as the addition of peptides.

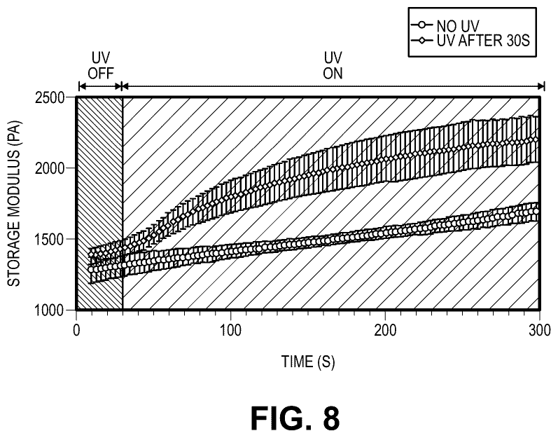

To anneal the microgels we added additional PEG-DT in a solution of HEPES with photoinitiator (FIG. 1B). The microgels were then spun down and supernatant removed. For larger microgels a higher centripetal force is required to effectively jam the microgels due to the increased voice space in between them. The aggregated microgels can be mixed with cells or other additives as the microgels exhibit shear thinning behavior and can easily be manipulated. The microgel slurry was plated and exposed to UV light to finish the annealing process. To determine necessary time for annealing, we measured the storage modulus of microgel slurries during UV exposure (FIG. 8). We found UV exposure at 20 mW cm² for 1 min significantly increased storage modulus, while 2 min resulted in a stronger annealing response. The increase in storage modulus in the control group without UV light can be attributed to slight annealing of the microgels through Michael Addition. This could be modulated by changing the pH of the annealing solution. Our approach is advantageous compared to other methods that may require hours for assembly.[21,23] Furthermore, the capacity to anneal microgels at room temperature with only a UV light source increases clinical accessibility. This strategy is amenable to the use of other photoinitiators sensitive to different wavelengths such as those found in the visible light spectrum (e.g., eosin Y).[17]

We created microgels with 50, 60, and 70% of the arms crosslinked to determine how many of the VS groups to leave for annealing. We tested the compressive modulus of scaffolds made from these microgels to observe how strongly they were annealed together. We also tested the addition of N-vinyl-2-pyrrolidone (NVP) which has been shown to increase gelation of PEG-VS by increasing the diffusion of free radicals. Microgels with 60% of the arms crosslinked with added PEG-DT resulted in the scaffold with the highest mechanical modulus (FIG. 1C). We hypothesize this is due to crosslinking enough arms to increase the mechanical moduli of a microgel while leaving enough arms free to anneal neighboring microgels. The group in just HEPES also exhibited a weak annealing response most likely due to the already present PEG-DT molecules. We found the addition of NVP reduced annealing kinetics perhaps due to sequestering some of the PEG-DT molecules. Based on these results the rest of our studies utilized microgels with 60% of the PEG-VS crosslinked. These were able to maintain their mechanical moduli while also leaving enough arms available for annealing. We also tested adding PEG-DT in several concentrations to our microgels with theoretical 4.8 mM concentration being the maximum we would need to fully crosslink all arms (FIG. 1D). While 2 mM resulted in a large portion of the microgels dissociating from the scaffold, 5 mM and 10 mM PEG-DT resulted in similar amounts of dissociation. Moving forward we utilized a 5 mM concentration of PEG-DT for annealing.

Finally, we verified that we could create microgel scaffolds that were of clinically relevant thickness (FIG. 1E). We fabricated microgel scaffolds that were up to 1 cm in thickness which is the depth that may be experienced in a full-thickness osteochondral lesion. This confirmed the microgels do not significantly attenuate UV light in large scaffolds. Furthermore, the 1 cm thick microgel scaffold was able to withstand cyclically applied compression, demonstrating the strength of the annealing. This method of annealing microgels was aimed to minimize the steps needed for scaffold formation. By creating microgels which only require a UV light source to crosslink the goal is to increase the accessibility of this platform. By stoichiometrically controlling the number of arms used for crosslinking and annealing we minimize the number of reagents needed to form an annealed microgel scaffold.

### Example 2—Mechanical Properties of Microgels and Cryopreservation

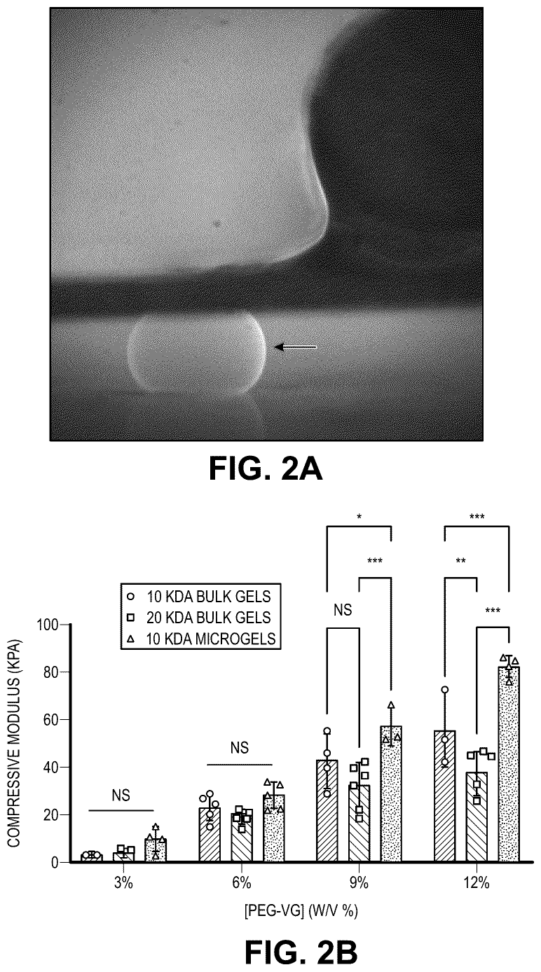

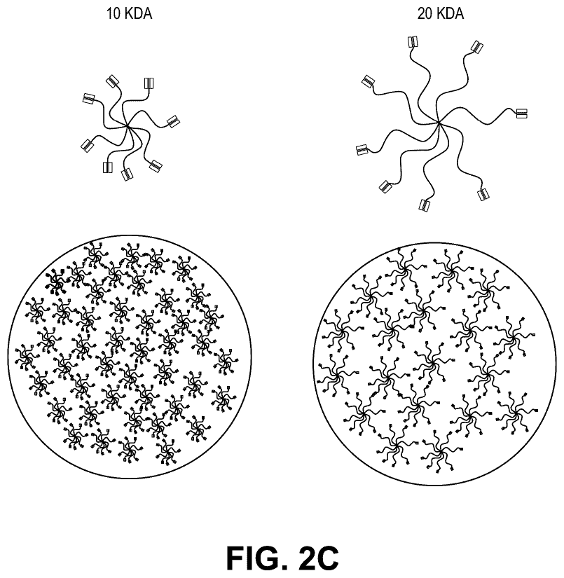

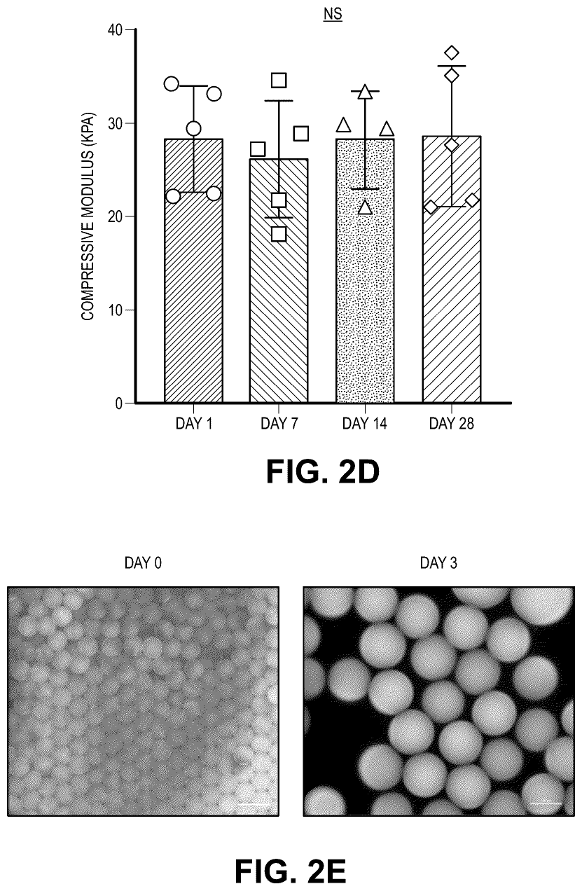

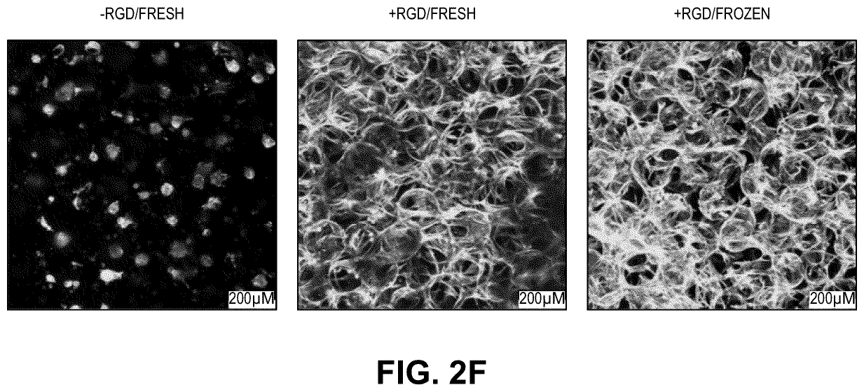

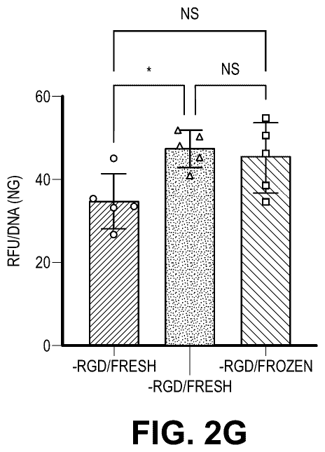

Understanding the mechanical properties of our microgels is important when designing scaffolds for specific tissues. Often it is ideal for scaffolds to match the mechanical moduli of the target tissue. There is a lack of research on how the microscale properties of a material compare to the macroscopic properties of its bulk counterpart. We utilized a MicroTester to test the compressive modulus of individual microgels (FIG. 2A). This compressive device allowed us to measure the mechanical modulus of individual microgels by measuring force and displacement of the microgel.

We compared the mechanical properties of our microgels to that of conventional bulk gels (FIG. 2B). All groups followed a similar trend of increased compressive modulus with increased concentration of PEG-VS as expected. We were able to generate microgels with stiffnesses ranging from about 10 kPa to about 82 kPa with macromer concentrations of 3% and 12%, respectively. A larger range of mechanical properties could be generated by lowering or raising the macromer concentration further. The modulus of the microgels were higher than their respective bulk gels in the 9% and 12% conditions. This could be due to the difference in testing methods where the microgels were tested on a MicroTester, and the microgels were tested on an Instron. Alternatively, this could be due to microgels being more homogenous than their bulk counter parts which often may not be uniformly smooth or crosslinked. The slight heterogeneity of bulk gels may result in a softer modulus. Between the 10 and 20 kDa bulk gel groups the 10 kDa trended toward a higher modulus as PEG-VS concentration increased. This is presumably due to the 10 kDa gels having a small mesh size (FIG. 2C). We continued experiments with the 10 kDa due PEG-VS going forward due to its stiffness having a large dynamic range.

The ability of the microgels to be cryopreserved is important for long-term storage and clinical application. We performed mechanical testing on 6% PEG-VS microgels frozen at −20° C. for up to a month and saw no change in mechanical properties (FIG. 2D). We also tested the ability of RGD-functionalized microgels to remain bioactive after being frozen for a month. We verified that we could biochemically functionalize our microgels by using fluorescent peptides. Peptides can be utilized to influence cell behavior such as attachment or differentiation. Both upon synthesis and after 3 days the fluorescent peptide was clearly present on the microgels (FIG. 2E). This verified that our washing and collection steps did not hinder the attachment of peptides. We saw a similar amount of cell spreading in human mesenchymal stromal cells (hMSCs) in both frozen and freshly made microgels compared to microgels with no added peptides (FIG. 2F). Interestingly, the cells in microgels without peptides formed spheroids most likely due to the lack of adhesive sites on the microgels. This was reflected with higher activity in an alamarBlue assay presumably due to spheroids being more metabolically active (FIG. 2G). However, all conditions demonstrated significant metabolic activity further indicating the microgels were bioactive after being frozen for a month.

By measuring the mechanical properties of the microgels and demonstrating their ability to be cryopreserved we further illustrated how they can be useful in the clinic. The large range of stiffnesses along with peptide bioactivity permit these microgels to be tailored for a variety of tissue types. The microgels demonstrated their ability to maintain a constant compressive modulus and peptide function after cryopreservation, opening the door for on-demand use in the clinic.

### Example 3—Modeling of Void Space in Matlab

Modeling the porosity of our microgel scaffolds is crucial for predicting how cells may migrate, aggregate, and differentiate within our constructs. Smaller microgels will have less void space in between them and therefore cells will be increasingly monodispersed with less room to spread out. Conversely, larger microgels will have increased void space in between them so cells will be able to more easily migrate and aggregate. Similarly, the more polydisperse microgels are the larger the void space will be due to the microgels being unable to optimally pack together. To model these phenomena we utilized custom MATLAB code along with FIJI to predict how actual or theoretical batches of microgels may pack together.

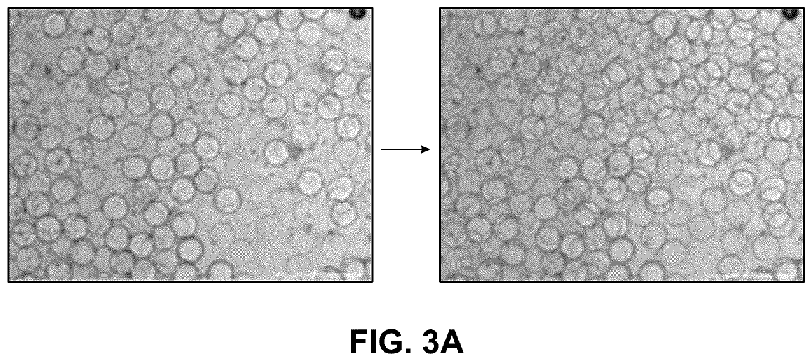

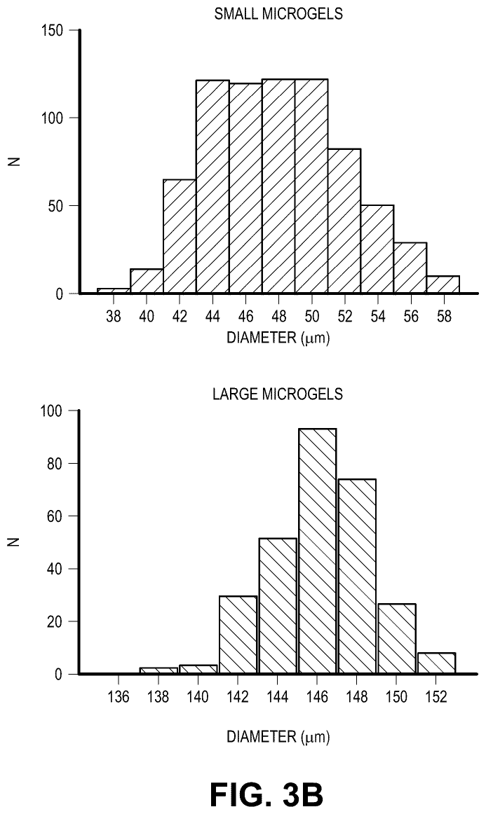

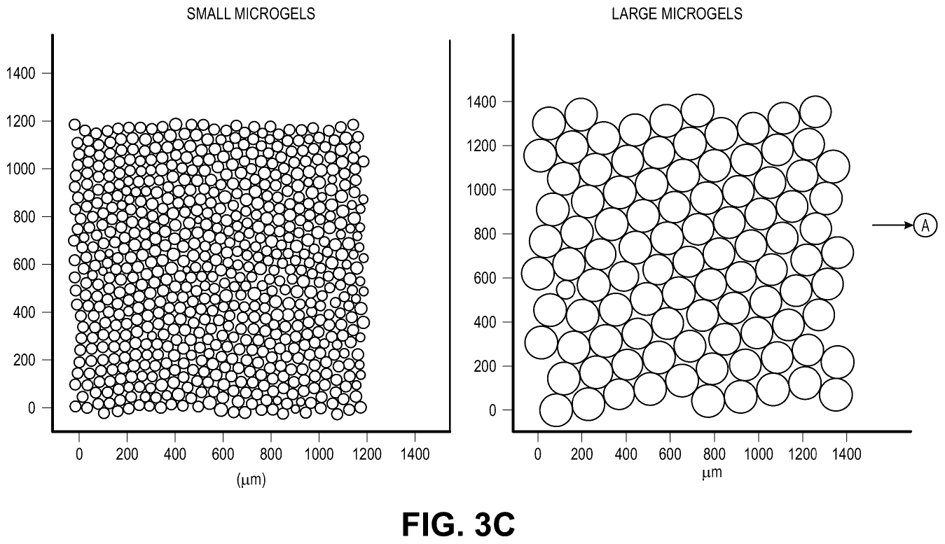

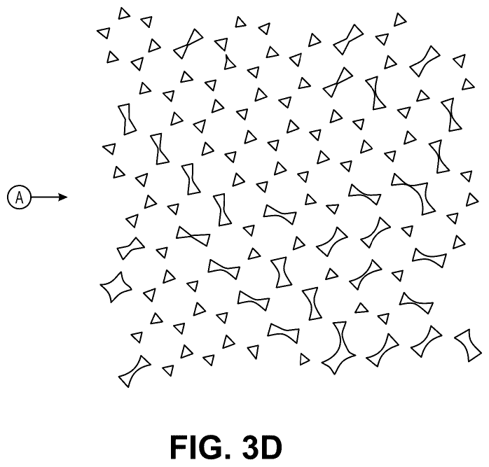

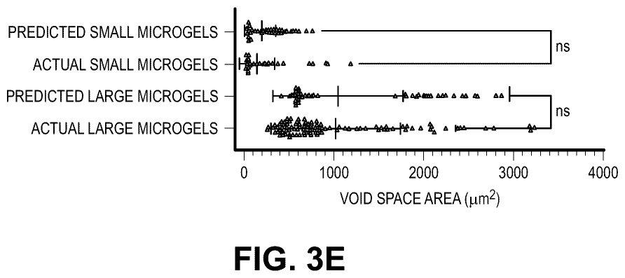

To simulate how an actual batch may pack together we first used MATLAB to identify and measure the diameters of batches of large and small microgels (FIG. 3A). The large batch of microgels had an average diameter of 146.1±2.68 μm while the small batch of microgels had an average diameter of 47.91±4.11 μm (FIG. 3B). The slightly larger standard deviation in the smaller batch could be contributed to the increased surface area to volume ratio of the smaller microgels requiring more surfactant. We then imported the measured diameters into a custom MATLAB script based on established code which will close pack the microgels and minimize the sum of distances between them (FIG. 3C). The microgels will converge to hexagonal close packing (HCP) the more monodisperse they are.

We utilized FIJI to calculate the area of the void space between the microgels (FIG. 3D). For the more monodisperse large microgels much of the void space area is triangular due to convergence to HCP, while some larger theoretical areas can be seen between the more polydisperse microgels. We validated our model experimentally and found that the actual average void space between microgels is not significantly different than our predicted area (FIG. 3E). Minor variance between the two could be attributed to the microgels slightly compressing or packing imperfectly. While actual diameters were used in the modeling a list of theoretical diameters could be inputted to predict what other void spaces may be.

The ability to model void space is beneficial for many cell-based projects which can vary greatly in the scale of biologics used. There are significant differences between cell types such as red blood cells and adipocytes which can range from 7.5 μm to over 100 μm in diameter, respectively. Aggregates of cells such as mesenchymal spheroids can reach up to 600 μm without a hypoxic core. It is also useful for the incorporation of other additives such as drug-loaded nanoparticles. Overall, modeling is a powerful tool that can be utilized to fabricate microgels for a specific porosity to influence cell phenotype.

### Example 4—Evaluation of Annealed Microgels to Promote Formation of Cell Aggregates

Multicellular spheroids can act as building blocks that capture complex aspects of in vivo environments and represent improved model systems to study development and disease. Spheroids exhibit increased viability along with enhanced proangiogenic, anti-inflammatory, and tissue-forming potential. Thus, the development of a platform that can promote the formation of cellular aggregates would be useful for many tissue engineering applications. To further investigate the impact of microgel size on cellular aggregate formation, we seeded monodisperse MSCs in small and large microgel scaffolds lacking adhesive moieties to encourage cell-cell adhesion and promote aggregate formation. We observed spheroid formation over the first 48 hours, which is a typical timeframe for spheroid formation using other methods such as hanging drop or formation in non-adhesive well plates.

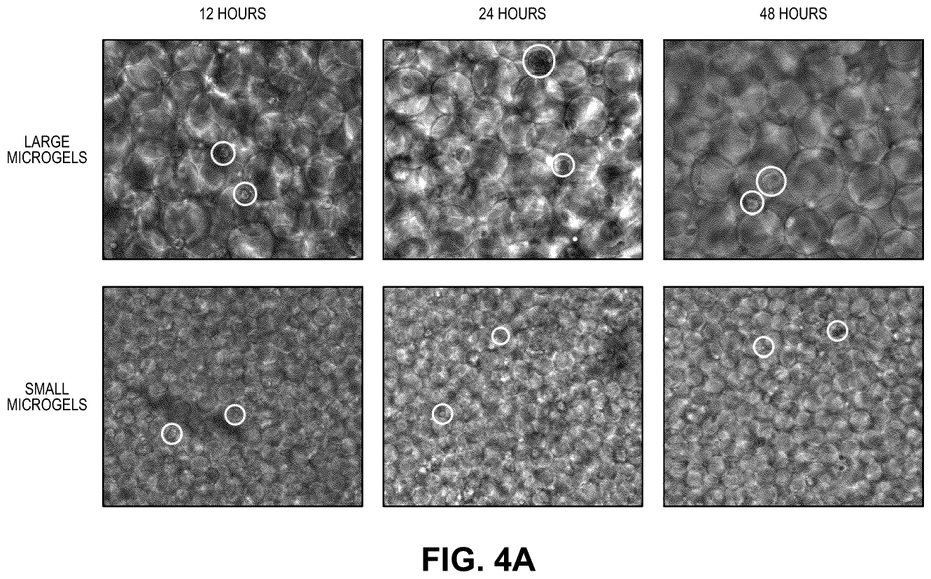

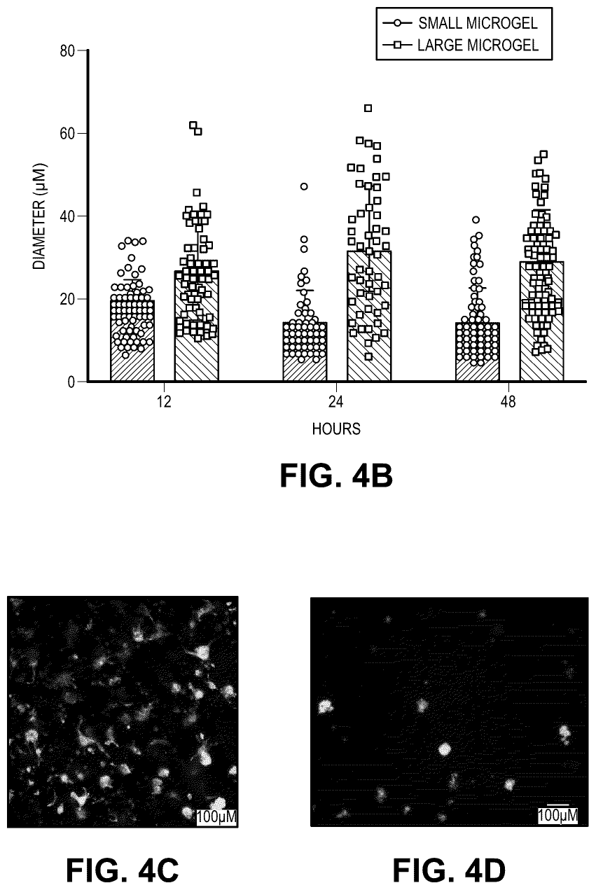

Aggregate formation was visible in large diameter microgel scaffolds after 12 hours, with monodisperse cells still present throughout the scaffold. The average aggregate size was consistent over 48 hours. MSCs in small diameter microgels formed smaller aggregates after 12 hours with many cells remaining monodisperse (FIG. 4A). We observed a trend towards a smaller aggregate diameter over 48 hours, which may be due to compaction of the aggregates. Quantification of average aggregate size over time illustrates how the void space in large microgel scaffolds promotes increased aggregate size compared to small microgel scaffolds (FIG. 4B). The average aggregate diameters at 48 hours in the large and small microgel scaffolds were ~32.0 μm and 14.8 μm, respectively, which strongly correlates with the observed void space areas in FIG. 3E.

Confocal microscopy of large diameter microgel scaffolds at 48 hours allows us to clearly visualize aggregates through the scaffold (FIG. 4C). While some cells remained monodisperse, the majority were contained in aggregates. We observed minimal cell spreading, possibly due to the deposition of extracellular matrix (ECM) by the MSCs. Furthermore, we were able to dissociate the microgels and retrieve viable cellular aggregates (FIG. 4D). For this application, we weakly annealed the microgels by not using NVP in our annealing solution, which permitted dissociation by simple pipetting. This demonstrates the potential for microgels to be used as a spheroid formation platform, where spheroids can be formed and collected for use in another medium. A scaffold which promotes spheroid formation may eliminate the need to form spheroids a priori, which commonly requires a minimum of 48 hours. Given the correlation between our model of void space and aggregate size, it would be possible to design specific size microgels for formation of a desired spheroid size.

### Example 5—Effects of Microgel Diameter on Cell Proliferation and Spreading

The void space between microgels allows for rapid cell infiltration and proliferation without cells needing to remodel the surrounding environment, as is required in bulk hydrogels. To assess how void space influences cell spreading, we seeded spheroids in scaffolds composed of large and small microgels. By seeding the microgels with spheroids, we can examine migration distance and density which is not possible if the cells were monodisperse. Microgels increase MSC retention and proliferation compared to traditional nanoporous hydrogels. However, the influence of altering microgel diameter on MSC growth has not been investigated. We hypothesized the increased surface area present in small microgel scaffolds would promote faster cell migration from the spheroid into the scaffold.

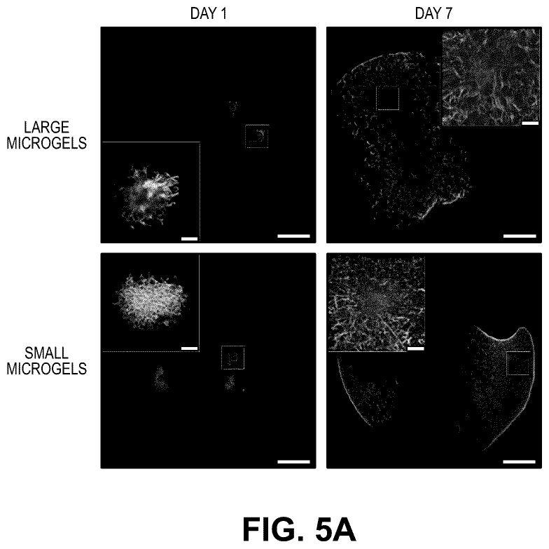

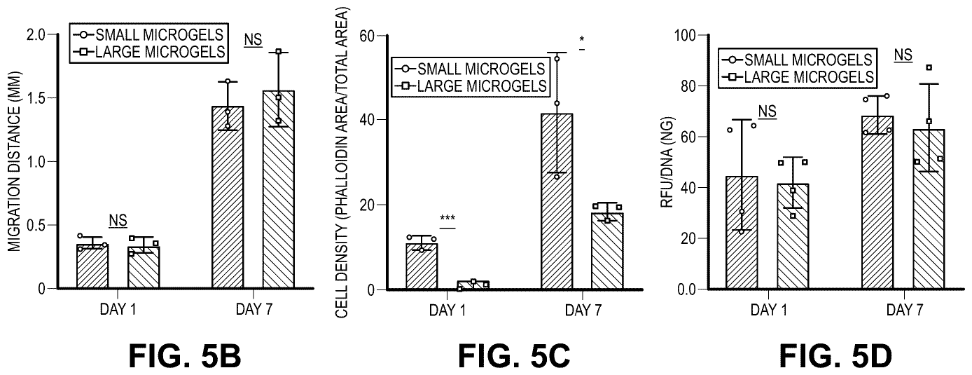

We seeded 15,000 cell spheroids composed of mesenchymal stromal cells (MSCs) and endothelial cells at a 2:1 ratio in our microgel scaffolds, as we previously demonstrated this spheroid composition forms robust cellular networks. Microgels were modified with RGD to promote cell adhesion and migration. We assessed network formation and migration distance on Day 1 and Day 7 via confocal microscopy and stained cells with DAPI and phalloidin to visualize the nuclei and actin cytoskeleton, respectively (FIG. 5A).

Surprisingly, both large and small microgels promoted rapid migration of cells into the scaffold. The leading edge of cell migration was comparable for both microgel sizes on Days 1 and 7 (FIG. 5B). The greater porosity within large microgel scaffolds did not significantly hinder cell migration. However, the smaller diameter microgel scaffold had a higher cell density on Days 1 and 7 (FIG. 5C). This can be attributed to the higher surface area-to-volume ratio of the smaller microgels that provide more attachment sites for cell spreading. Both scaffolds promoted similar levels of metabolic activity when normalized to DNA, indicating both formulations promote high viability (FIG. 5D).

The rapid migration of cells in both conditions highlights the advantage of inherent porosity in microgel scaffolds. The measurable migration on Day 1 reflects the cells ability to immediately migrate without first remodeling the surrounding environment. While void space size is significantly smaller in small microgel scaffolds compared to large microgel scaffolds, it was sufficient to permit cell movement. Future work could utilize different size microgels to regulate the density of cell infiltration and spreading.

### Example 6—Effects of Microgel Size on Macrophage Polarization

Solid biomaterial implants often induce a foreign body response, regulated by macrophages, that is characterized by poor vascularization and fibrosis. Ideally, a biomaterial will promote a pro-regenerative response characterized by cell infiltration and material integration. Microgels can promote a pro-regenerative M2 phenotype compared to clinical controls such as Oasis Wound Matrix decellularized ECM. However, the impact of altering microgel size and void space on macrophage polarization has not been studied.

We seeded IC-21 macrophages in 4.5% PEG-VS large and small microgel scaffolds modified with 1 mM RGD to enable attachment. We chose this medium stiffness formulation to minimize the effect of substrate stiffness on macrophage polarization. Macrophages were collected after 6 days in culture for assessment of polarization via flow cytometry. We utilized our “weak” annealing formulation lacking NVP described previously to facilitate macrophage recovery from the gels. A subset of macrophages was stained with CellTrace to permit visualization with fluorescent microscopy and observe their interaction with the microgel scaffolds.

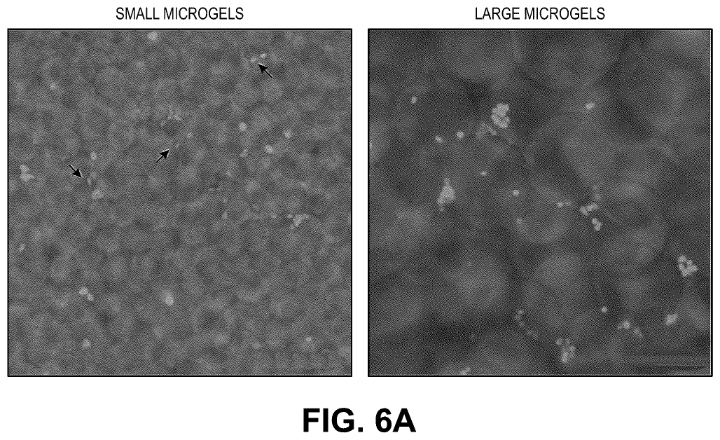

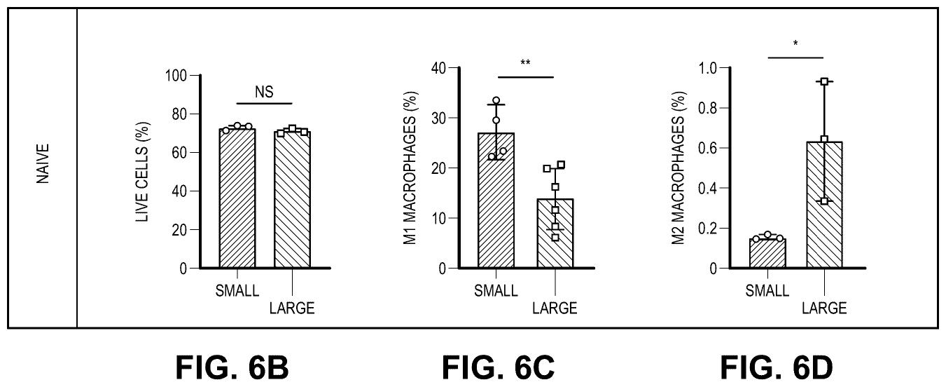

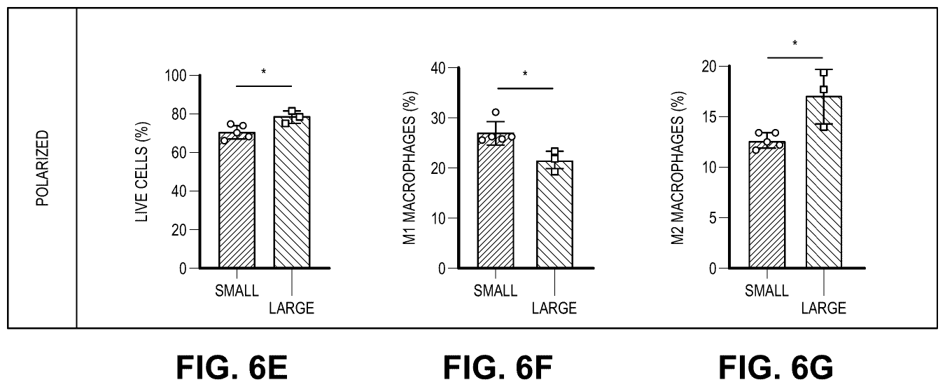

Confocal microscopy of the macrophages revealed that in small microgel scaffolds, macrophages were often sandwiched between individual microgels, with several exhibiting an elongated morphology. Conversely, macrophages easily fit in the void spaces of large microgel scaffolds and generally maintain a rounded morphology (FIG. 6A). In some cases, clusters of macrophages can be seen that were not present in the small microgel scaffold.

Flow cytometry revealed that both microgels supported high cell viability (FIGS. 6B and 6E). Macrophages with an M1 phenotype (F4/80+CD86+iNOS+ populations) were more prevalent in small microgel scaffolds with both naïve and polarized macrophages (FIGS. 6C and 6F). Macrophages with an M2 phenotype (F4/80+CD206+ARG1+ populations) accounted for significantly more of the macrophages in the large microgel scaffold in both macrophage conditions (FIGS. 6D and 6G). These observations agree with earlier work wherein minimizing macrophage adhesion to implants upregulates the M2 phenotype.[39,41,44] We also show similar trends exist between naïve and M1 polarized macrophages. This is relevant to wound or surgical sites whereM1macrophages are typically associated. The increased void space between large microgels limits the amount of contact macrophages have with the scaffold, often with macrophages contacting only one microgel. Conversely, in the small microgel scaffold, macrophages often are contactin gmultiple microgels at once and are stimulated from all sides.

By increasing the porosity, it may be possible to reduce the foreign body response (FBR) to a material and increase the presence of pro-regenerative M2 macrophages. Microgels are a promising candidate for porous biomaterials given the tunable void space that exists between them. While the porosity in the small microgel scaffold was large enough to promote rapid and dense spreading as demonstrated previously, it may be so small as to promote unintended effects such as a pro-inflammatory response from macrophages. Therefore, when picking a microgel size, it is important to consider the resultant porosity between them.

### Example 7—Microgel Scaffolds Permit Endogenous Cell Infiltration In Vivo

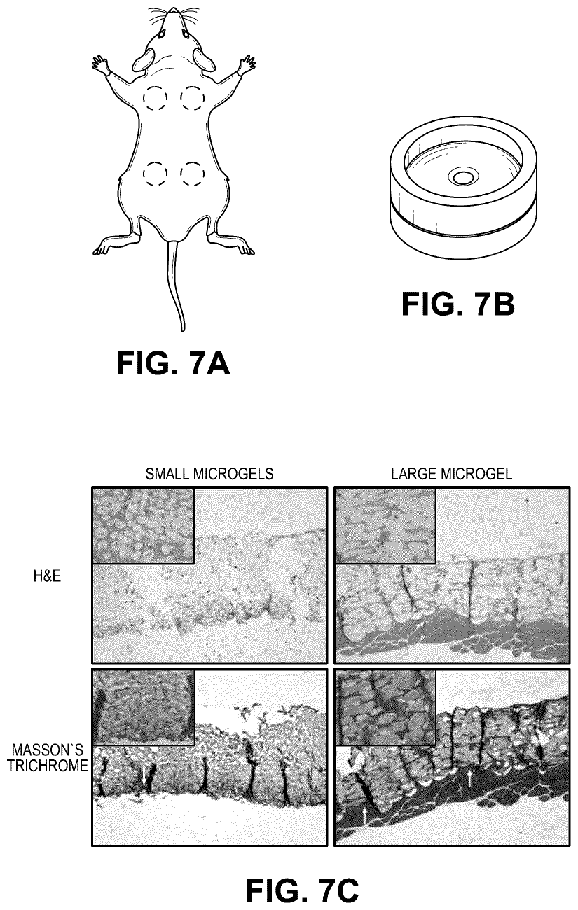

We interrogated how cellular infiltration in vivo would be influenced by different size microgel scaffolds. We implanted small and large microgel scaffolds subfascially in C57BL/6 mice for 2 weeks to assess endogenous cell migration (FIG. 7A). We used PDMS molds to prevent infiltration from one side of the scaffold to accurately assess migration from the other side (FIG. 7B). Implants were harvested after 2 weeks for histological processing.

Hematoxylin and eosin (H&E) staining revealed that while cells infiltrated both scaffolds, they tended to migrate farther inside the larger microgel scaffolds (FIG. 7C). Where cells did infiltrate, they surround both sizes of microgels which indicates the porosity in both scaffolds was sufficient for migration. The reduced migration depth in the smaller microgel scaffolds could be a result of the smaller porosity hindering migration or the increased surface area resulting in cells spreading out more densely but not as far. Masson's trichrome staining corroborated the increased migration seen in the large microgel scaffolds (FIG. 7C). Larger aggregates of cells are present between the larger microgels. Notably, collagen is present primarily around the surface of the implants but is scarce in between the microgels themselves. While this indicates a FBR to the PDMS mold in which the microgel scaffolds were housed, it appears the FBR to the microgels themselves was minimized.

In vivo implantation resulted in robust endogenous cell spreading and infiltration in our microgel scaffolds. The increased surface area of the smaller microgels resulted in denser spreading near the surface of the scaffold, but less migration into the scaffold. Conversely, cellular aggregates in the larger microgel scaffold consistently migrated the depth of the scaffold but were more spread out. This study demonstrates microgels as a promising biomaterial to promote rapid cell infiltration and biomaterial integration. Furthermore, these data further illustrate the importance of pore size and its direct effect on cell density and spreading.

### Example 8—Experimental Protocols

Device Fabrication: Soft lithography was used to create microfluidic master molds on silicon wafers (University Wafer) for devices first described in Rutte et al. We used a two-layer photolithography process with SU-8 10 and SU-8 100 (Kayaku Advanced Materials) to create channels heights for our different sized microgels. The layers were aligned utilizing a EVG 620-mask aligner. A Bruker Dektak XT was used to verify the heights of our device channels. A nozzle channel height of 12 μm and 38 μm created droplets of ~48 μm and 146 μm, respectively. Polydimethylsiloxane (PDMS) (Ellsworth Adhesives, Sylgard 184) was then poured over our silicon master molds with the base and crosslinker mixed at a 10:1 mass ratio. The PDMS mixture was desiccated and cured at 65° C. for at least an hour and cut out. PDMS devices and glass slides were plasma cleaned and bonded together followed by a bake of 125° C. on a hotplate for 1 hour. The microgel devices were then treated with Aquapel and Novec 7500 Oil (3M) to render the devices hydrophobic and fluorophilic, respectively.

Microgel Fabrication: Our microgel devices contained a continuous oil phase and a dispersed aqueous phase. The oil phase consisted of Novec 7500 Oil and 0.75% wt Picosurf (Sphere Fluidics) for the large microgels. Picosurf concentration was increased to 2% wt for the small microgels. The aqueous phase consisted of 8-arm PEG-VS (JenKem) in 0.15 M triethanolamine (TEOA, pH 5.1, Sigma) buffer and 3.5 kDa PEG-DT (JenKem). The solutions were injected into the microfluidic using syringe pumps (NE-1000 and World Precision Instruments) with the continuous phase set at twice the flow rate of the dispersed phase. We utilized flow rates of 60/30 μL/min for the large microgels and 30/15 μL/min for the small microgels. After exiting the device microgels were combined with a solution of 1% v/v triethylamine (TEA, Sigma)) in Novec 7500 Oil using a Y-junction (IDEX Health and Science). The microgels were left at room temperature overnight to ensure complete crosslinking.

Microgel Cleaning: To clean the microgels first excess oil was removed by pipetting. A solution of 20 wt % 1H,1H,2H,2H-Perfluoro-1-octanol (Sigma) in Novec 7500 Oil approximately equal to the volume of remaining microgels was then added to break the emulsion. HEPES buffer (25 mM, pH 7.4) was then added to swell and disperse the microgels. A hexane wash was then repeated 3× to remove the remaining oil. Enough hexane was added to pellet the microgels at the bottom after being spun down at 2000 g. For cell experiments the microgels were then washed 3× with 70% ethanol for sterilization. Finally, the microgels were washed with sterile HEPES buffer 3× where they remained until use.

Microgel Annealing: To anneal the microgels they were first spun down at 2000 g and excess liquid removed. The microgels were then resuspended in a solution of 5 mM PEG-DT in HEPES containing 0.4% VA-086 photoinitiator (FUJIFILM) equal to the volume of microgels. After incubating for at least 1 minute, the microgels were spun down for 3 minutes at 3000 g for the small microgels and 15,000 g for the larger microgels. The supernatant was then removed and microgels plated in the desired mold utilizing a positive displacement pipette (Gilson). The microgel slurry was then exposed to UV light (20 mW/cm2, Omnicure S2000) for 2 minutes to form annealed scaffolds.

Annealing Optimization Experiments: Microgels were created such that 60,70, and 80% of the PEG-VS arms were crosslinked with PEG-DT. Microgels were then soaked in a solution containing 0.4% photoinitiator in HEPES, HEPES+PEG-DT, HEPES+NVP (Sigma), or HEPES+PEG-DT+NVP. Microgel scaffolds were then formed as detailed previously and mechanically testing was performed and described below.

Formation of Large Microgel Constructs: To form 1 cm tall cylindrical molds we first poured PDMS into a petri dish until it was 1 cm tall. After curing we then used an 8 mm biopsy punch to create an 8 mm×1 cm cylindrical mold in the PDMS. Microgels were then pipetted and annealed via the previously described methods.

Mechanical Characterization: To mechanically characterize the bulk hydrogel scaffolds we used an Instron 3345 Compressive Testing System (Norwood, MA). Hydrogels were loaded between two flat platens and compressed at a rate of 0.05 mm/s. Moduli were calculated from the slope of stress versus strain plots limited to the linear first 10% of strain. To characterize the compressive modulus of the microgels were utilized a MicroTester (CellScale). Individual microgels were loaded onto an anvil in a water bath filled with PBS. The microgels were then compressed half their diameter by a stainless steel platen attached to a tungsten rod over 30 s. Displacement and force was tracked via the proprietary MicroTester software. The compressive modulus was calculated over the course of compression using formulas described in Kim et al. The linear region of the of compressive modulus vs nominal strain graph was recorded as the calculated modulus. Storage modulus of microgel scaffolds over time was measured using a Discovery HR2 Rheometer (TA Instruments, New Castle, DE) with a stainless steel, cross hatched, 8 mm plate geometry. For the experimental group, the scaffolds were exposed to UV light after 30 s, while it remained off the entire time for the control group. A custom oscillatory time sweep (1% strain, 1 rad s¹ angular frequency) was performed using an initial 0.03N normal force.

Peptide Verification: To verify we were successfully adding peptides to our microgels we added 2 mM fluorescently tagged HAVDI (SEQ ID NO: 16) (FITC-HAVDIGGGC (SEQ ID NO: 17), WatsonBio) to our microgels. Microgels were immediately imaged upon synthesis as well as 3 days post-synthesis and washing.

Cryogenic Studies: 6% PEG-VS large microgels with 2 mM RGD (Ac-RGDSPGERCG-NH2 (SEQ ID NO: 15), Genscript) were utilized in the cryogenic studies. Microgels were frozen for up to a month with batches being taken out of the freezer at designated time points. Mechanical testing was performed on the MicroTester. After being frozen for a month microgels were seeded with hMSCs (RoosterBio, Frederick, MD) at a density of 1 million cells/mL and compared to fresh microgels. An alamar blue (ThermoFisher) assay was performed to assess metabolic activity after 2 days. A 2×10−3 M Calcein AM (ThermoFisher) solution was added to visualize cells. Gels were imaged to assess bioactivity after a 30 minute incubation.

Cell culture: Human endothelial colony forming cells (ECFCs) were isolated and derived from human cord blood obtained through the UC Davis Cord Blood Collection Program (UCBCP). ECFCs were expanded in EGM-2 supplemented media (PromoCell, Heidelberg, Germany) with gentamicin (50 μg mL⁻¹; ThermoFisher, Waltham, MA) and amphotericin B (50 ng mL⁻¹; ThermoFisher) under standard culture conditions (37° C., 5% CO₂, 21% O₂) until use at passages 7-8. Media changes were performed every 2 days. Human bone marrow-derived MSCs (RoosterBio) from a single donor (21-year-old male) were expanded in growth medium (GM) consisting of minimum essential alpha medium (α-MEM; Invitrogen) supplemented with 10% fetal bovine serum (FBS; Genesee) and 1% penicillin/streptomycin (Gemini Bio-Products, Sacramento, CA). MSCs were cultured under standard conditions until use at passage 4. Media changes were performed every 2-3 days.

Influence of cryostorage on microgels: We fabricated 6% PEG-VS large diameter microgels with 1 mM RGD (Ac-RGDSPGERCG-NH2 (SEQ ID NO: 15), Genscript). Microgels were frozen for up to one month at −20° C., with batches being removed, thawed, and analyzed at designated time points. Mechanical testing was performed on the MicroTester as described above. After 1 week in storage, microgels were seeded with human MSCs (RoosterBio) at 5 million cells mL⁻¹ and compared to fresh microgels when maintained in complete medium for 48 hr. Metabolic activity of seeded MSCs was determined by alamarBlue assay (Thermo Fisher). The cell actin cytoskeleton was stained with Alexa Fluor 488 Phalloidin solution (Thermo Fisher; 1:400 in PBS), and cell nuclei were stained with DAPI (Thermo Fisher; 1:500 in PBS). Z-stacks were taken on a confocal microscope (Leica Stellaris 5), and max projections used to illustrate cell morphology through the scaffolds.

Void Space Modeling: A custom MATLAB code was used to measure microgel diameters and model annealing. FIJI was used to measure the void space area between microgels.

Formation of cell aggregates within scaffolds: Small and large microgel scaffolds composed of 6% PEG-VS were seeded with MSCs at 5 million cells mL⁻¹. Images were acquired at 12, 24, and 48 hr with brightfield microscopy (Nikon Eclipse TE2000U). Aggregates from large microgels were collected by pipetting the scaffolds up and down, followed by pipetting the solution through a 100 μm sieve. Aggregates were washed with PBS, stained with a live/dead assay per the manufacturer's protocol (Thermo Fisher), and fluorescent images taken using the Nikon Eclipse TE2000U. For confocal microscopy images, cells were stained and imaged with DAPI and phalloidin as described above. Aggregate size was measured in ImageJ using the line and measurement tools.

Assessment of spreading in scaffolds from co-culture spheroids: Heterotypic co-culture spheroids were formed with ECFCs and MSCs at a ratio of 1-to-2 using a forced aggregation method. Briefly, desired concentrations of ECFCs and MSCs were pipetted into 1.5% agarose molds in well plates, and the plates were centrifuged at 500×g for 8 min. Plates were maintained in static standard culture conditions (37° C., 5% CO₂, 21% O₂) for 48 hr to enable spheroid formation in 3:1 EGM-2:α-MEM. Each microwell in the agarose molds contained 15,000 cells. 4.5% PEG-VS microgels with 1 mM RGD were used for the large and small microgel scaffolds. Microgels were annealed as described above, with the microgel slurry being mixed with heterotypic spheroids before being plated in a 6 mm×1.5 mm cylindrical silicon mold and exposed to UV light. Scaffolds were collected for analysis on Day 1 and Day 3, with media changed every other day using the 3:1 mixture of EGM-2:α-MEM. For confocal microscopy images, cells were stained and imaged with DAPI and phalloidin as described above. Metabolic activity of spheroids was determined by alamarBlue assay (Thermo Fisher). DNA content was quantified using the PicoGreen Quanit-iT Assay Kit (Invitrogen). Migration distance and cell density were quantified in ImageJ using the Li threshold.

Assessment of macrophage polarization as a function of microgel diameter: IC-21 murine macrophages (ATCC) were seeded at 4 million cells mL⁻¹ in small or large microgel scaffolds. Microgels were formed from 4.5% PEG-VS with 1 mM RGD prepolymer solution. Microgel and macrophage slurries were pipetted into 8 mm×5 mm cylindrical molds before exposure to UV light. The gels were maintained in RPMI 1640 (ATCC) supplemented with 10% FBS. After 24 hr, gels were moved to a new plate with fresh media and maintained in culture for 6 days with media changes every day. For confocal microscopy, 8 mm×1.5 mm scaffolds were prepared and imaged on the Leica Stellaris 5. Cells were recovered from annealed patties via digestion at 37° C. with trypsin for 5 min and gentle mixing, followed by dilution with basal media. Solutions were filtered through a 30 μm sieve to create a single cell suspension, and macrophage polarization was characterized using flow cytometry (Attune NxT, Life Tech).

Flow cytometry: Following Fcγ receptor blocking (1:40, TruStain FcX, BioLegend), cells were stained with antibodies against F4/80 (1:50, eBioscience #MF48021), CD86 (1:160, eBioscience #47-0862-82) and CD206 (1:40, eBioscience #48-2061-82). Cellular viability was evaluated with fixable Zombie Aqua (1:250, Life Tech). Cells were then fixed with 2% PFA, permeabilized with 0.1% Triton-X, and stained for intracellular markers, iNOS (1:500, eBioscience #12-5920-82) and Arginase-1 (1:500, eBioscience #53-3697-82), overnight at 4° C. with gentle agitation. Macrophages with an M1 phenotype were characterized by F4/80+CD86+iNOS+ populations and M2 phenotypes by F4/80+CD206+ARG1+ populations. The frequency of each type of macrophage was quantified per microgel size. Polarization controls (data not shown) consisted of IC-21s seeded on TC wells in monolayer treated with basal media (M0), 200 ng/mL LPS (M1), and 20 ng/mL IL-4 (M2) for 24 hr. Cells were lifted with trypsin and gentle agitation. Cells were filtered and stained as described.

Subfascial Implants: Before implantation, 4.5% PEG-VS microgels with 1 mM RGD were loaded and annealed in PDMS molds. Treatment of experimental animals was in accordance with UC Davis animal care guidelines and all National Institutes of Health animal handling procedures. Male twelve-week-old C57BL/6 mice (Jackson Laboratories, West Sacramento, CA) were anesthetized and maintained under a 2% isoflurane/02 mixture delivered through a nose cone. Each animal received four subfascial implants: small microgels (upper and lower left) and large microgels (upper and lower right). Following a dorsal midline incision, fascia was incised, and blunt dissection was performed between the fascia and muscle belly. Annealed microgels in PDMS molds were placed face-down on the muscle and sutured in place with 4-0 Monocryl sutures (Ethicon, Cornelia, GA). Animals were euthanized after 2 weeks, and gels were collected, removed from PDMS, and fixed in 4% PFA overnight at 4° C. Samples were then washed twice in PBS, paraffin-embedded, and sectioned at 7 μm. Sections were stained with hematoxylin (Thermo) and eosin (Ricca) (H&E) or Masson's trichrome (Sigma) and imaged using an EVOS XL Core (Invitrogen).

Statistical Analysis: All statistics were calculated using GraphPad Prism 9. Statistical significance was assessed by either one-way ANOVA, two-way ANOVA with Tukey's multiple comparisons test, two-way ANOVA with Sidak's multiple comparisons test, or Student's t-test when appropriate. p-values<0.05 were considered statistically significant.

### REFERENCES

[1] S. M. V. Belleghem, B. Mahadik, K. L. Snodderly, J. P. Fisher, In Biomaterials Science, Elsevier, 2020, pp. 1289-1316.
[2] N. Ashammakhi, S. Ahadian, M. A. Darabi, M. El Tahchi, J. Lee, K. Suthiwanich, A. Sheikhi, M. R. Dokmeci, R. Oklu, A. Khademhosseini, Adv. Mater. 2019, 31, 1804041.
[3] J. M. Lowen, J. K. Leach, Adv. Funct. Mater. 2020, 30, 1909089.
[4] A. C. Daly, L. Riley, T. Segura, J. A. Burdick, Nat. Rev. Mater. 2020, 5, 20.
[5] A. S. Caldwell, B. A. Aguado, K. S. Anseth, Adv. Funct. Mater. 2020, 30, 1907670.
[6] B. N. Pfaff, L. J. Pruett, N. J. Cornell, J. de Rutte, D. Di Carlo, C. B. Highley, D. R. Griffin, ACS Biomater. Sci. Eng. 2021, 7, 422.
[7] D. R. Griffin, W. M. Weaver, P. O. Scumpia, D. Di Carlo, T. Segura, Nat. Mater. 2015, 14, 737.
[8] T. H. Qazi, J. Wu, V. G. Muir, S. Weintraub, S. E. Gullbrand, D. Lee, D. Issadore, J. A. Burdick, Adv. Mater. 2022, 2109194.
[9]S. Xin, C. A. Gregory, D. L. Alge, Acta Biomater. 2020, 101, 227.
[10]Q. Feng, D. Li, Q. Li, X. Cao, H. Dong, Bioact. Mater. 2022, 9, 105.
[11]T. H. Qazi, J. A. Burdick, Biomater. Biosyst. 2021, 1, 100008.
[12]M. P. Lutolf, J. L. Lauer-Fields, H. G. Schmoekel, A. T. Metters, F. E. Weber, G. B. Fields, J. A. Hubbell, Proc. Natl. Acad. Sci. U.S.A. 2003, 100, 5413.
[13]B. P. James, W. Sicheng, P.-H. Qiang, Q.-J. Marianne, FEMS Immunol. Med. Microbiol. 2005, 45, 159.
[14]J. M. de Rutte, J. Koh, D. Di Carlo, Adv. Funct. Mater. 2019, 29, 1900071.
[15]C. Ji, A. Khademhosseini, F. Dehghani, Biomaterials 2011, 32, 9719.
[16]N. Annabi, J. W. Nichol, X. Zhong, C. Ji, S. Koshy, A. Khademhosseini, F. Dehghani, Tissue Eng., Part B 2010, 16, 371.
[17]E. Sideris, D. R. Griffin, Y. Ding, S. Li, W. M. Weaver, D. Di Carlo, T. Hsiai, T. Segura, ACS Biomater. Sci. Eng. 2016, 2, 2034.
[18]A. S. Caldwell, G. T. Campbell, K. M. T. Shekiro, K. S. Anseth, Adv. Healthcare Mater. 2017, 6, 1700254.
[19]N. F. Truong, E. Kurt, N. Tahmizyan, S. C. Lesher-Perez, M. Chen, N. J. Darling, W. Xi, T. Segura, Acta Biomater. 2019, 94, 160.
[20]H. Lv, L. Li, M. Sun, Y. Zhang, L. Chen, Y. Rong, Y. Li, Stem Cell Res. Ther. 2015, 6, 103.
[21]L. R. Nih, E. Sideris, S. T. Carmichael, T. Segura, Adv. Mater. 2017, 29, 1606471.
[22]J. E. Mealy, J. J. Chung, H. Jeong, D. Issadore, D. Lee, P. Atluri, J. A. Burdick, Adv. Mater. 2018, 30, 1705912.
[23]Q. Feng, Q. Li, H. Wen, J. Chen, M. Liang, H. Huang, D. Lan, H. Dong, X. Cao, Adv. Funct. Mater. 2019, 29, 1906690.
[24]J. R. Day, A. David, J. Kim, E. A. Farkash, M. Cascalho, N. Milasinovid, A. Shikanov, Acta Biomater. 2018, 67, 42.
[25]Y. Hao, H. Shih, Z. Munoz, A. Kemp, C.-C. Lin, Acta Biomater. 2014, 10, 104.
[26]C. F. Guimaraes, L. Gasperini, A. P. Marques, R. L. Reis, Nat. Rev. Mater. 2020, 5, 351.
[27]K. Hosoyama, C. Lazurko, M. Mu5 oz, C. D. McTiernan, E. I. Alarcon, Front. Bioeng. Biotechnol. 2019, 7, 205.
[28]T. Xu, Random close packing (RCP) on arbitrary distribution of circle sizes, https://github.com/BluesBlues213/random-close-packing, accessed: April, 2022.
[29]M. Diez-Silva, M. Dao, J. Han, C.-T. Lim, S. Suresh, MRS Bull. 2010, 35, 382.
[30]M. B. Ginzberg, R. Kafri, M. Kirschner, Science 2015, 348, 1245075.
[31]K. C. Murphy, B. P. Hung, S. Browne-Bourne, D. Zhou, J. Yeung, D. C. Genetos, J. K. Leach, J. R. Soc., Interface 2017, 14, 20160851.
[32]M. Fathi-Achachelouei, H. Knopf-Marques, C. E. Ribeiro da Silva, J. Barthes, E. Bat, A. Tezcaner, N. E. Vrana, Front. Bioeng. Biotechnol. 2019, 7, 113.
[33]M. A. Gionet-Gonzales, J. K. Leach, Biomed. Mater. 2018, 13, 034109.
[34]K. C. Murphy, S. Y. Fang, J. K. Leach, Cell Tissue Res. 2014, 357, 91.
[35]T. Gonzalez-Fernandez, A. J. Tenorio, J. K. Leach, 3D Print. Addit. Manuf 2020, 7, 139.
[36]C. E. Vorwald, S. S. Ho, J. Whitehead, J. K. Leach, In Biomaterials for Tissue Engineering (Ed.: Chawla, K.), Springer New York, New York, NY, 2018, pp. 139-149.
[37]J. Koh, D. R. Griffin, M. M. Archang, A. Feng, T. Horn, M. Margolis, D. Zalazar, T. Segura, P. O. Scumpia, D. Carlo, Small 2019, 15, 1903147.
[38]C. E. Vorwald, S. Joshee, J. K. Leach, J. Mol. Med. 2020, 98, 425.
[39]B. D. Ratner, Regener. Biomater. 2016, 3, 107.
[40]Y. Liu, T. Segura, Front. Bioeng. Biotechnol. 2020, 8, 609297.
[41]L. J. Pruett, C. H. Jenkins, N. S. Singh, K. J. Catallo, D. R. Griffin, Adv. Funct. Mater. 2021, 31, 2104337.
[42]M. Haschak, S. LoPresti, E. Stahl, S. Dash, B. Popovich, B. N. Brown, Aging 2021, 13, 16938.
[43]M. Chen, Y. Zhang, P. Zhou, X. Liu, H. Zhao, X. Zhou, Q. Gu, B. Li, X. Zhu, Q. Shi, Bioact. Mater. 2020, 5, 880.
[44]E. M. Sussman, M. C. Halpin, J. Muster, R. T. Moon, B. D. Ratner, Ann. Biomed. Eng. 2014, 42, 1508.
[45]J. Whitehead, K. H. Griffin, M. Gionet-Gonzales, C. E. Vorwald, S. E. Cinque, J. K. Leach, Biomaterials 2021, 269, 120607.
[46]T. Gonzalez-Fernandez, A. J. Tenorio, A. M. Saiz Jr, J. K. Leach, Adv. Healthcare Mater. 2022, 11, 2102337.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A microgel scaffold comprising:
a plurality of polyethylene glycol-vinyl sulfone (PEG-VS); and
a plurality of polyethylene glycol-dithiol (PEG-DT) covalently linked to the plurality of PEG-VS,
wherein the microgel scaffold has a compressive modulus of between 1 kPa and 30 kPa.

2. The microgel scaffold of claim 1, wherein the microgel scaffold has a porosity of between 20 μm² and 100000 μm².

3. A microgel scaffold comprising:
a plurality of polyethylene glycol-vinyl sulfone (PEG-VS); and
a plurality of polyethylene glycol-dithiol (PEG-DT) covalently linked to the plurality of PEG-VS,
wherein the microgel scaffold has a porosity of between 20 m² and 100000 μm².

4. The microgel scaffold of claim 3, wherein the microgel scaffold has a compressive modulus of between 1 kPa and 30 kPa.

5. The microgel scaffold of any one of claims 1 to 4, wherein the PEG-VS is selected from the group consisting of 3-arm PEG-VS. 4-arm PEG-VS, 6-arm PEG-VS, and 8-arm PEG-VS.

6. The microgel scaffold of any one of claims 1 to 5, wherein the PEG-VS has a molecular weight ranging from between 2 kDa and 40 kDa.

7. The microgel scaffold of any one of claims 1 to 6, wherein the PEG-DT has a molecular weight ranging from between 0.6 kDa and 35 kDa.

8. The microgel scaffold of any one of claims 1 to 7, between 40% and 80% of the PEG-VS are linked to PEG-DT.

9. The microgel scaffold of any one of claims 1 to 8, wherein the microgel scaffold further comprises a plurality of functional moieties conjugated to the PEG-VS.

10. The microgel scaffold of claim 9, wherein the functional moiety is a peptide.

11. The microgel scaffold of any one of claims 1 to 10, further comprising live cells seeded within the microgel scaffold.

12. The microgel scaffold of claim 11, wherein the cells comprise human mesenchymal stem cells, induced pluripotent stem cells, embryonic stem cells, osteoblasts, immune cells, blood cells, adipocytes, or combinations thereof.

13. A method of generating a microgel scaffold, the method comprising:
forming a reaction mixture comprising a polyethylene glycol-vinyl sulfone (PEG-VS) and a polyethylene glycol-dithiol (PEG-DT);
exposing the reaction mixture to ultraviolet (UV) irradiation for less than 2 minutes to anneal the PEG-VS and the PEG-DT, thereby forming the microgel scaffold.

14. The method of claim 13, wherein the PEG-VS is selected from the group consisting of 3-arm PEG-VS, 4-arm PEG-VS, 6-arm PEG-VS, and 8-arm PEG-VS.

15. The method of claim 13 or 14, wherein the PEG-VS has a molecular weight ranging from between 2 kDa and 40 kDa.

16. The method of any one of claims 13 to 15, wherein the concentration of the PEG-VS in the reaction mixture is between 1 mM and 80 mM.

17. The method of any one of claims 13 to 16, wherein the PEG-DT has a molecular weight ranging from between 0.6 kDa and 35 kDa.

18. The method of any one of claims 13 to 17, wherein the concentration of the PEG-DT in the reaction mixture is between 1 mM and 300 mM.

19. The method of any one of claims 13 to 18, wherein between 40% and 80% of the PEG-VS are linked to PEG-DT.

20. The method of any one of claims 13 to 19, wherein prior to the exposing step, a photoinitiator is added to the reaction mixture.

21. The method of claim 20, wherein the concentration of the photoinitiator in the reaction mixture is between 0.05% w/v and 0.8% w/v.

22. The method of any one of claims 13 to 21, wherein the UV irradiation has a power within the range between 2 mW/cm² and 20 mW/cm².

23. The method of any one of claims 13 to 22, further comprising, after the exposing step, attaching a functional moiety to the microgel scaffold.

24. The method of claim 23, wherein the functional moiety is a peptide.

25. The method of any one of claims 13 to 24, further comprising, after the exposing step, centrifuging the microgel scaffold at different speeds to achieve a desired porosity of the microgel scaffold.

26. The method of any one of claims 13 to 25, further comprising, after the exposing step, seeding the microgel scaffold with live cells.

27. The method of claim 26, wherein the cells comprise human mesenchymal stem cells, induced pluripotent stem cells, embryonic stem cells, osteoblasts, immune cells, blood cells, adipocytes, or combinations thereof.


SEQUENCE LISTING (17 sequences)

SEQ ID NO: 1 - protein, 10 aa, synthetic construct
RGDSPGERCG

SEQ ID NO: 2 - protein, 9 aa, synthetic construct
HAVDIGGGC

SEQ ID NO: 3 - protein, 10 aa, synthetic construct
LNIVSVNGRH

SEQ ID NO: 4 - protein, 9 aa, synthetic construct
DNRIRLQAK

SEQ ID NO: 5 - protein, 17 aa, synthetic construct
KATPMLKMRTSFHGCIK

SEQ ID NO: 6 - protein, 21 aa, synthetic construct
KEGYKVRLDLNITLEFRTTSK

SEQ ID NO: 7 - protein, 21 aa, synthetic construct
KNLEISRSTFDLLRNSYGVRK

SEQ ID NO: 8 - protein, 22 aa, synthetic construct
KQNCLSSRASFRGCVRNLRLSR

SEQ ID NO: 9 - protein, 22 aa, synthetic construct
KQKCLRSQTSFRGCLRKLALIK

SEQ ID NO: 10 - protein, 22 aa, synthetic construct
CRNRGRCNSSLFQVRSRKLLSA

SEQ ID NO: 11 - protein, 22 aa, synthetic construct
KQCLKSQRSFTRGLCRLKAKIL

SEQ ID NO: 12 - protein, 15 aa, synthetic construct
KLLISRARKQAASIK

SEQ ID NO: 13 - protein, 16 aa, synthetic construct
LERKYENDQKYLEDKA

SEQ ID NO: 14 - protein, 10 aa, synthetic construct
VEKRGDREEA

SEQ ID NO: 15 - protein, 10 aa, synthetic construct
  SITE 1: note = Acetylated residue
  SITE 10: note = Amidated residue
RGDSPGERCG

SEQ ID NO: 16 - protein, 5 aa, synthetic construct
HAVDI

SEQ ID NO: 17 - protein, 9 aa, synthetic construct
  SITE 1: note = Fluorescein isothiocyanate modified residue
HAVDIGGGC